(19) United States
(12) Patent Application Publication
(10) Pub. No.: US 2025/0017646 A1
(43) Pub. Date: Jan. 16, 2025

(54) PERCUTANEOUS TREATMENT DEVICE AND METHOD

(71) Applicant: Pulse Biosciences, Inc., Hayward, CA (US)

(72) Inventors: Kevin L. MOSS, Lathrop, CA (US); Cameron D. HINMAN, Thurmond, NC (US); Bryan DUENAS, San Jose, CA (US); David J. DANITZ, San Jose, CA (US)

(21) Appl. No.: 18/701,211

(22) PCT Filed: Sep. 15, 2022
(86) PCT No.: PCT/US2022/076507

§ 371 Date: Apr. 12, 2024

Related U.S. Application Data
(60) Provisional application No. 63/271,954, filed on Oct. 26, 2021.

(51) Int. Cl. A61B 18/14; A61B 17/00; A61B 18/00

(52) U.S. Cl. CPC A61B 18/148; A61B 2017/00194; A61B 2018/00077; A61B 2018/00083; A61B 2018/00601; A61B 2018/0091; A61B 2018/1415; A61B 2218/002; A61B 2218/007

(57) ABSTRACT

Described herein are percutaneous treatment tools and methods for applying electric treatment to a target tissue, hi some examples the treatment tools described herein allow for adjusting the spacing between the proximal electrode(s) and the distal electrode of the treatment tools. The treatment tools described herein may also be configured to reduce peak electric field for a given potential and/or to increase hoop stress on the tissue upon insertion of the tip to prevent or reduce arcing between the electrodes.

### CLAIM OF PRIORITY

This patent application claims priority to U.S. provisional patent application No. 63/271,954, titled “PERCUTANEOUS TREATMENT DEVICE AND METHOD,” filed on Oct. 26, 2021, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Various electrical devices, including in the form of radio frequency (RF), microwave, cryo, laser or pulsed electric energy delivery devices, are commonly used for treating certain conditions and diseases. For example, electric pulses have been described for electromanipulation of biological cells. Electric pulses, including short, high-field strength electric pulses, may be used in treatment of human cells and tissue including benign and malignant tumor cells, lesions, various tissue and skin growth and conditions. Treatments with electric pulses, including higher electric field strengths and shorter electric pulses, may be useful in manipulating intracellular structures, such as nuclei and mitochondria. For example, sub-microsecond (e.g., nanosecond) high voltage pulse generators and treatment applicators have been proposed for biological, medical and cosmetic applications. However, such higher peak electric fields are more likely to are between the electrodes.

It would be particularly advantageous to be able to treat various tissue and anatomical structures of a subject with electric fields using percutaneous approach, for example, with a percutaneous needle-type apparatus or tool. However, delivering therapeutic high voltage energy has a substantial risk inducing electrical shock, arcing, burns, internal-organ damage, risks that are even more acute when the high voltage device is intended to be inserted into the body, for example, percutaneously. Because of the complexity of this challenge, no known effective devices have been developed.

It would be beneficial to provide devices, such as treatment apparatuses, especially percutaneous treatment apparatuses (e.g., tools), and corresponding methods that may apply high voltage, electrical pulses, such as sub-microsecond electric pulses, to treat patients while mitigating these risks.

### SUMMARY OF THE DISCLOSURE

Described herein are apparatuses (e.g., systems, devices and tools, including applicators) and methods for treating various anatomical structures using electrical fields. In general, these apparatuses and methods may be useful for treating a subject by the application of therapeutic energy, including but not limited to short, high field strength electric pulses, such as pulses having sub-microsecond (e.g., nanosecond) duration. The systems, devices and methodologies described herein are especially useful with non-thermal pulsed electric fields (e.g., nanosecond pulsed electric fields, etc.), however, in some implementations they can be also used with other energy modalities, including (but not limited to) radio frequency (RF) energy. The devices, systems and methods described herein are also configured to avoid or reduce arcing, especially when applying high voltage electric fields.

The methods and apparatuses (e.g., devices, systems, etc.) described herein may apply sub-microsecond (e.g., nanosecond) pulsed electrical fields to treat lesions, tumors, nodules and other growth, diseases and conditions, for example, in a target tissue, including various anatomical structures. Such anatomical structures may include muscular organs (e.g., smooth muscle, cardiac and skeletal muscles), circulatory organs (e.g., heart, arteries, veins), respiratory organs (e.g., lungs), abdomen and digestive organs (e.g., stomach, duodenum, intestines, liver), urinary organs (e.g., kidneys, ureters, bladder), immune system organs (e.g., lymph nodes, bone marrow, thymus), nervous system organs (e.g., brain, spinal cord, nerves), endocrine organs (e.g., pituitary gland, thyroid, adrenal glands), reproductive organs (e.g., penis, vagina, prostate, uterus, testicle), skeletal organ (e.g., bones).

In some examples, the apparatuses, e.g., treatment applicators, described herein may provide easy percutaneous access to a target tissue, such as thyroid, and perform electric field treatments of the affected target tissue, for example, treatment of the thyroid nodule. The disclosed apparatuses allow for less trauma to the target tissue and also allow to adjust the length/depth and size of the area being treated by adjusting a distance between the electrodes of the apparatus. The apparatuses described herein may be used with a variety of different generator systems, for example, nanosecond pulse generators.

The apparatuses described herein may be configured for manual or automated (e.g., robotic assisted) or semi-automated control and may be particularly well suited for use with various fully and partially automated systems, such as robotic systems. In some variations these apparatuses may be integrated into systems that are configured to be mounted onto or coupled to a movable (e.g., robotic) arm of a robotic system. In some variations instruments can be guided and controlled by the robotic system during a medical or cosmetic procedure.

For example, described herein are apparatuses for delivering an electric field treatment that include: a handle; an elongate shaft extending from the handle; a tip region at a distal end of the elongate shaft, the tip region comprising: a first electrode, a second electrode, and a spacer between the first electrode and the second electrode; and a length adjuster configured to adjust a distance between the first electrode and the second electrode.

Any of these apparatuses may be configured to include a vacuum outlet at the distal end (tip region) that may assist in making contact between the tissue and electrodes. For example, the apparatus may include a vacuum channel configured to provide a negative pressure at the tip region. Alternatively or additionally, the apparatus may include an infusion channel configured to deliver a solution from the tip region. The tip region may include a vacuum outlet or an infusion outlet, e.g., disposed between the first electrode and the second electrode. In some examples the same channel may be used for inflation as for vacuum.

The electrodes may be electrically coupled to a pulse generator by one or more connections in the handle. These connections may be configured to prevent leak current (e.g., creepage) within the handle, which may be particularly important when high voltages are used, as may be the case with sub-microsecond pulsing in some treatment regimens. For example, the apparatus may include a first wire connecting to the first electrode and a second wire connecting to the second electrode. These connections may be isolated from each other and from within the handle. For example, in some examples, the handle comprises an insulating baffle configured to provide a minimum clearance distance between electrical contacts for the first electrode and the second electrode within the handle.

In any of these apparatuses the first electrode may be proximal to the second electrode along the tip region. For example, the first electrode may be a circumferential electrode and the second electrode may be configured with a tissue-penetrating distal end.

The spacer may be conductive or insulative. In some examples it may be particularly beneficial to include a conductive spacer between the first and second (or between first, second and third or more) electrodes. The spacer may have a circumference that is greater than a circumference of either of the first electrode and the second electrode. The circumference may refer to the outer dimension of the spacer or outer radial dimension, in some examples the circumference may refer to the outer diameter (OD); in general the circumference does not need to be round but may have any shape.

The spacer between the first and second electrode may also be configured to reduce arcing by providing a long minimum clearance distance (e.g., minimum creepage path distance) between the first electrode and the second electrode. For example, the spacer may be coupled to the proximal end of the second (distal) electrode and may be configured so that the minimum clearance distance or path from the second electrode to the first (proximal) electrode is along the spacer. The spacer may also be configured so that the minimum clearance distance/path is greater than the minimum distance between the first electrode and the second electrode (e.g., the distance between the first and second electrodes on the outside of the device). In some examples, the spacer may extend proximally from a proximal end of the second electrode and proximal to the first electrode; the spacer may also extend radially inwards of the first electrode.

In any of the apparatuses described herein the apparatus may include a first elongate member to which the first electrode is coupled, and a second elongate member concentrically within the first elongate member and to which the second electrode is coupled. The first elongate member may form the outer portion of the shaft or may be housed within the shaft. Thus, in some examples, the first elongate member may form at least a portion of the elongate shaft.

The length adjuster may be configured to drive movement (e.g. axial movement in the proximal-to-distal direction) of the first elongate member relative to the second elongate member. For example, the length adjuster may include a threaded body configured to convert rotational movement of an outer portion of the length adjuster into linear movement of the second elongate member relative to the first elongate member to move the second electrode relative to the first electrode to adjust the distance between the first electrode and the second electrode. In some examples the length adjuster comprises an adjuster knob configured to drive movement of a stator coupled to the second elongate member. The adjuster knob may be configured to rotate clockwise or counterclockwise to drive the stator proximally or distally without rotating the stator.

In any of these methods and apparatuses, the distance between the first electrode and the second electrode may be adjustable, for example, from 1 mm to 7 mm. In any of these examples adjusting the distance between the first and second electrodes may also adjust a spacer between the first and second electrodes. In some examples adjusting the spacing between the first and second electrodes may cause the outer circumference (e.g., the outer diameter) of the spacer to expand or contract; however in some examples the spacer outer circumference may be adjusted separately from the electrode spacing.

Thus, in any of these apparatuses and methods the spacer may be stretched or compressed, which may reduce or increase the outer circumference (e.g., diameter) of the spacer. In some examples it may be beneficial to reduce arcing to have the spacer have an outer diameter (OD) that is greater than either the OD of the first or second electrodes. However, it may also be beneficial to insert the apparatus into the tissue with a more uniform outer diameter (e.g., low profile circumference) without the spacer having an OD extending further than the ODs of the first or second electrodes. In any of the methods and apparatuses the spacer may be configured so that the circumference (e.g., OD) of the spacer may be reduced (e.g., during insertion) and expanded to a larger circumference (e.g., OD), for example, when the apparatus is in place for the application of energy. For example, the spacer may be formed of an elastomeric material that has an expanded configuration with an OD that is larger than the ODs of either the first or second electrodes. The proximal end of the spacer may be coupled to a member that may pull (and/or push) the spacer to compress it, so that the OD of the spacer is reduced, e.g., for insertion. Alternatively, the spacer may be in a normally lower profile configuration (e.g., which may be the same as or less than the OD of the first and/or second electrode), and the proximal end may be pushed to compress the spacer so that the OD expands to a larger dimension than the OD of both the first and second electrodes.

In some examples of the apparatuses described herein the second electrode comprises a tissue-penetrating end, which may be configured as a trocar, a cone or a hybrid of a trocar and a cone or other smooth surface shape.

Any of the apparatuses described herein may include a pulse generator configured to generate a plurality of electrical pulses having amplitude of at least 0.1 kV and a duration of less than 1000 nanoseconds.

In any of these apparatuses described herein, each of the first electrode and the second electrode may include a curved edge (referred to herein as a fillet) on each side of the first electrode and the second electrode facing the spacer.

For example, an apparatus for delivering a pulsed electric field may include: a handle; an elongate shaft extending from the handle; a tip region extending distally from the elongate shaft, the tip region comprising: a first electrode, a second electrode, and a conductive spacer between the first electrode and the second electrode.

Thus, an apparatus for delivering high voltage electric field may be configured to reduce peak electric field. The apparatus may comprise a handle and a tip region coupled to the handle, the tip region comprising a first electrode, a second electrode, and a spacer between the first electrode and the second electrode, wherein the first electrode comprises at least one first fillet on a first side adjacent to the spacer and the second electrode comprises at least one second fillet on a second side adjacent to the spacer such that the at least one first fillet and the at least one second fillet are configured to reduce or eliminate arcing between the first and the second electrode. In some examples, the fillets are configured to reduce peak electric field by, for example, about 10% to about 30%. The spacer between the electrodes may be insulating or in some examples it may be a conductive spacer. In some examples the spacer may be a balloon. Also, in some examples, the spacer may have a circumference (e.g. diameter) the same or greater than a circumference of the electrodes. In further examples, the circumference of the spacer near the middle of the spacer may be greater than the circumferences of the electrodes while each end of the spacer adjacent to the electrodes may have the same circumference as the electrodes, and the circumference of the spacer may gradually reduce in the direction from the middle of the spacer towards each end. The apparatus may further comprise a length adjuster configured to adjust distance between the first electrode and the second electrode. In some examples, one of the electrodes that is at a distal end of the tip region may have a trocar configuration, a conical configuration (“cone” or “pencil”), or a hybrid configuration. In some embodiments, the electrodes may be bipolar, which in other embodiments the electrodes may be monopolar.

For example, an apparatus for delivering a pulsed electric field may include: a handle; an elongate shaft extending from the handle; and a tip region extending distally from the elongate shaft, the tip region comprising: a first electrode, a second electrode, and a spacer between the first electrode and the second electrode, wherein the first electrode comprises a first rounded edge on a first side of the first electrode that is adjacent to the spacer and the second electrode comprises a second rounded edge on a second side of the second electrode adjacent to the spacer, wherein the first rounded edge and the second rounded edge are configured to reduce or eliminate arcing between the first electrode and the second electrode.

The apparatus for delivering a pulsed electric field may comprise a handle and a tip region coupled to the handle, wherein the tip region comprises a first electrode, a second electrode, and a conductive spacer between the first electrode and the second electrode.

The circumference (e.g., outer circumference) of the conductive spacer may be same as the circumference of each of the first electrode and the second electrode. In some examples, the circumference of the conductive spacer may be greater than a circumference of the electrodes. For example, the circumference of the conductive spacer near a middle portion of the conductive spacer may be greater than a circumference of the first electrode and the second electrode, and each end of the conductive spacer adjacent to the first electrode and the second electrode may have the same circumference as the circumference of the first electrode and the second electrode, further wherein the circumference of the conductive spacer tapers from a middle of the conductive spacer towards each end of the conductive spacer. The circumference of the conductive spacer may be adjusted depending on the shape of the target region and/or the treatment requirement of the applied electric field.

In some examples, the electrodes adjacent to the conductive spacer may have fillets (rounded corners). In some examples, the second electrode is at a distal end of the tip region and this distal electrode may have a trocar configuration, a conical configuration, or a hybrid configuration.

The circumference of the conductive spacer may be adjusted depending on the shape of the target region and/or the treatment requirement of the applied electric field.

In some embodiments, the conductive spacer may be (or may include) a hydrogel, a conductive adhesive, a conductive gel, a conductive silicone, a urethane rubber, conductive thermoset, thermoplastic resins, any other biocompatible material with the desired conductivity, any semi-conductor material, or any combination thereof.

In some embodiments, a conductivity of the conductive spacer is substantially equal to ten times (10×) a conductivity of a tissue of the treatment area. In some embodiments, a conductivity of the conductive spacer is greater than or equal to a conductivity of a tissue of the treatment area and less than or equal to one hundred times (100×) the conductivity of the tissue of the treatment area.

The present disclosure is also directed to methods of using any of the apparatuses described herein, including selecting an electrode assembly. In some examples, selecting the electrode assembly may be based on the conductive spacer having a conductivity that is less than, greater than, or equal to a conductivity of a tissue of the treatment area. In further examples, the method may include selecting the electrode assembly based at least in part on a conductivity of the conductive spacer. Also, selecting the electrode assembly may be further based at least in part on a size of the electrode assembly and a size of the treatment area. In some examples, the method of treating a tissue with a pulsed electric field may include selecting the voltage to be applied to the treatment area based at least in part on a conductivity of the conductive spacer.

In some embodiments, the conductive spacer can reduce the peak electric field by 25%-50% compared to the electrodes with the insulative spacer. The conductive spacer may have another advantage of providing more uniform treatment between the electrodes by strengthening electric field in the middle between the electrodes of the tip region.

In some examples the apparatus for delivering electric treatment includes a handle and a tip region coupled to the handle, the tip region comprising a first electrode, a second electrode, and a spacer between the first electrode and the second electrode, wherein a maximum circumference of the spacer is greater than a maximum circumference of the first electrode and the second electrode.

As mentioned above, in some examples the distal end of the tips described herein may be configured as an electrode having a tissue-penetrating shape. The shape may be a cone (e.g. pencil) shape, or in some examples a trocar shape, having multiple cutting edges. In some example a hybrid of the cone and trocar shapes may be used. For example, an apparatus for delivering electric treatment may include a tip region extending distally from the elongate shaft, the tip region comprising: a first electrode, a second electrode distal to the first electrode, wherein the second electrode comprises a tissue-penetrating distal end configured as a hybrid of a cone and a trocar; and a spacer between the first electrode and the second electrode. For example, the second electrode may include a distal trocar region having three or more blade edges extending proximally from a distal end and a proximal conical region having a smooth conical face extending proximally from a proximal end of each blade. The change in angle between the three or more blade edges and the smooth conical face may be less than 15 degrees (e.g., less than about 15 degrees, less than about 14 degrees, less than about 13 degrees, less than about 12 degrees, less than about 11 degrees, less than about 10 degrees, etc.).

In some examples, the tip region may have any combination of the features described herein, including any of the spacers, electrode fillets (rounded edges) and any configuration of the distal electrode (e.g., cone, trocar, hybrid), as well as vacuum and/or infusion outlets (if applicable) and length adjustment capabilities. For example, in some embodiments, the tip region may have a trocar tip and a conductive spacer with the same circumference as electrodes. In another example, the tip region may have a cone/pencil tip and a conductive spacer with the same circumference as electrodes. This type of the tip region may mitigate arcing by both decreasing the peak electric field and also forcing the tissue to stretch during insertion improving the contact with the tissue. Also, in any of the examples of the present disclosure, the apparatus may be configured to provide an effective minimum clearance distance to avoid or reduce arcing without increasing the actual physical distance between the electrodes.

As mentioned above, in any of the apparatuses and methods described herein the spacer may extend proximally of the proximal (e.g., first) electrode, which may increase the minimum clearance/creepage distance between the first and second (e.g., distal) electrodes. The proximal electrode, and at least part of the insulated first electrical connector connecting the proximal electrode to the pulse generator, may be coaxially arranged over the insulated second electrical connector (e.g., wire) connecting the distal (e.g., second) electrode to the pulse generator. The proximal electrode may also be coaxially positioned at least over a portion of the spacer. In some examples the spacer extends proximally past the distal end of the proximal electrode further along at least a portion of the length of the proximal electrode (or a full length of the proximal electrode, or 1.25× the length of the proximal/first electrode, 1.5× the length of the first electrode, 1.75× the length of the first electrode, 2× the length of the first electrode, etc.). The spacers may generally be configured to prevent or reduce arcing and may be used (or formed) without the use of an adhesive/glue material, as in some cases the use of adhesive to bond and/or form the spacer may result in entrapped air bubbles that may result in arcing. Thus, in any of these apparatuses the spacer may be formed between the first and second electrodes without the use of an adhesive.

The apparatus for delivering a pulsed electric field described herein may comprise a handle and a tip region coupled to the handle (e.g., through an elongate shaft), the tip region may include a proximal electrode, a middle electrode, a tip electrode, a proximal spacer between the proximal electrode and the middle electrode, and a tip spacer between the middle electrode and the tip electrode. In some embodiments, the electrodes can be configured as bipolar or monopolar. In case of the bipolar operation, a proximal electrode and a tip electrode may be a positive electrode and a middle electrode may be a negative electrode, or the proximal electrode and the tip electrode may be the negative electrode and the middle electrode may be the positive electrode. Both proximal spacer and a tip spacer can be an insulative spacer or a conductive spacer, or one of the proximal spacer and the tip spacer can be the insulative spacer and the other of the proximal spacer and the tip spacer can be the conductive spacer.

Also described herein are systems for providing electric treatment. For example, the systems may include: an apparatus according to any examples of the present disclosure and a pulse generator configured to generate a plurality of electrical pulses having amplitude of at least 0.1 kV and a duration of less than 1000 nanoseconds.

As stated above, methods of using any of the apparatuses described herein are also included. For example, described herein are methods of treating a tissue with a sub-microsecond pulsed electric field comprising: inserting a tip region of a treatment tool into a target tissue, wherein the tip region comprises a first electrode that is proximal to a tissue-penetrating second electrode at a distal end of the tip region; adjusting a length adjuster on a proximal handle of the treatment tool to adjust a proximal-to-distal distance between the first electrode and the second electrode; and applying a plurality of electrical pulses having an amplitude of greater than 0.1 kV and a duration of less than 1000 nanoseconds, to treat the target tissue.

The proximal-to-distal distance between the first electrode and the second electrode may be adjusted based on a size of the target tissue. In any of these methods, adjusting the length adjuster may comprise rotating an adjuster knob of the length adjuster clockwise or counterclockwise. Any of these methods may include adjusting the proximal-to-distal distance between the first electrode and the second electrode between 1 mm and 7 mm.

As mentioned, any of these methods may include applying suction through a vacuum channel and a vacuum outlet of the treatment tool to secure the target tissue against the first electrode and the second electrode. Thus, any of these methods may include applying suction through a vacuum channel and a vacuum outlet to secure the target region of the tissue against the first electrode and the second electrode. Alternatively or additionally, the methods may include infusing a solution, e.g., a saline solution, through an infusion channel and an infusion outlet. The methods may include infusing a therapeutic agent to advance treatment by combining the electric pulse treatment and another therapeutic treatment.

The methods described herein may include: inserting, percutaneously, a tip region of a treatment tool into a target tissue, the tip region comprising a first electrode proximal to a second electrode; and applying a plurality of electrical pulses having a pulse duration of less than 1000 nanoseconds while reducing peak electric field for a given potential and/or increasing hoop stress on the target tissue to prevent or at least reduce arcing between the first electrode and the second electrode.

Any of these methods may improve contact with the tissue and/or reduce the likelihood of arcing by adjusting the position and/or dimensions of the spacer between the electrodes. For example, described herein are method that include: inserting a tip region of a treatment tool into a target tissue, wherein the tip region comprises a first electrode that is proximal to a tissue-penetrating second electrode at a distal end of the tip region; radially expanding a spacer between the first electrode and the second electrode so that a circumference (e.g., diameter) of the spacer is greater than a respective circumference (e.g., diameter) of both the first electrode and the second electrode to reduce arcing; and applying a plurality of electrical pulses having an amplitude of greater than 0.1 kV and a duration of less than 1000 nanoseconds, to treat the target tissue. The spacer may be in the first, narrow-diameter configuration when inserted into the tissue and may be expanded once positioned and before applying the energy. The spacer may be expanded by controlling a driver (e.g., plunger, pusher, puller, etc.) that may be on the handle of the device. Expanding the spacer may increase the contact with the tissue between the electrodes.

In general, the methods described herein may be methods of treating a target with a sub-microsecond pulsed field. Any of these methods may be methods of positioning the apparatus and/or methods of avoiding arcing, while creating an improved contact with the treatment area.

The methods described herein include methods of treating a thyroid module. For example, a method comprises inserting percutaneously a tip region of a treatment tool into a target thyroid nodule, the tip region comprising a first electrode and a second electrode; and applying through the first electrode and the second electrode a plurality of electrical pulses having a pulse duration of less than 1000 nanoseconds to thyroid nodule while reducing peak electric field for a given potential and/or increasing hoop stress on the thyroid nodule to prevent or at least reduce arcing between the first electrode and the second electrode. Thyroid nodule may be a benign thyroid nodule.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates an example of a system for delivering a high voltage, fast pulsed electrical energy.

FIG. 2 shows a perspective view of an example of an apparatus, configured as a treatment tool as described herein.

FIG. 3A shows a cross-sectional view of an example of a treatment tool with a vacuum and/or infusion tubing. FIG. 3B is a perspective view of an example of an enlarged tip region that can be used with the treatment tool of FIG. 3A.

FIG. 3C is a cross-section through an example of a treatment tool as described herein.

FIG. 3D is a cross-section through another example of a treatment tool as described herein in which the spacer extends proximally past the proximal electrode.

FIG. 4 shows a cross-sectional view of one example of a handle of a treatment tool with an adjustable treatment length.

FIGS. 5A-5B show examples of a treatment applicator having an adjustable treatment length. FIG. 5A shows an example of the length adjustment mechanism and FIG. 5B shows an example of a tip region with the adjustable spacing between the electrodes.

FIGS. 6A-6E show examples of handles of treatment tools/applicators as described herein. FIG. 6A shows an example of a pencil-style handle. FIG. 6B shows an exploded view of the example of FIG. 6A. FIG. 6C illustrates a side cross-sectional view of another example of a handle of a treatment tool similar to that shown in FIGS. 6A-6B. FIG. 6D shows an exploded perspective view of the handle of FIG. 6C, and FIG. 6E is an enlarged view of a portion of the exploded view of FIG. 6D.

FIGS. 7A-7E show examples of electric field distributions corresponding to the treatment tools having different features as described herein. FIG. 7A shows an example of baseline electric fields without various features of the present disclosure. FIG. 7B shows an example of an electric field distribution of a treatment tool having fillets (rounded corners). FIG. 7C shows an example of an electric field distribution of a treatment tool having a conductive spacer between the electrodes. FIG. 7D1 illustrates an example of a treatment tool having fillets on the electrodes and an insulative spacer with an outer diameter (OD)/circumference larger than the OD (e.g., circumference) of the electrodes, and FIG. 7D2 illustrates the corresponding electric fields distribution for the treatment tool of FIG. 7D1. FIG. 7E illustrates electric field distributions corresponding to a treatment tool having a conductive spacer with the same OD as the electrodes and fillets on the electrodes. In FIGS. 7A-7C and 7D1-7E the scale shown on the right for the heat map extends from 1 to 10 and has been normalized to a relative scale based on the baseline (this scale does not reflect an actual voltage).

FIGS. 8A-8B show a front view and a side view, respectively, of an example of an apparatus having a trocar tip.

FIGS. 8C-8D show a front view and a side view, respectively, of an example of a treatment applicator having a conical tip as described herein.

FIGS. 8E-8F show a front view and a side view, respectively, of an example of a hybrid tip according to the present disclosure.

FIGS. 9A-9F show examples of tip regions of apparatuses as described herein. FIG. 9A illustrates an example of a tip region having a hybrid tip and a spacer with the same circumference as electrodes. FIG. 9B illustrates an example of a tip region having a trocar tip and a spacer with a circumference greater than the electrodes. FIG. 9C illustrates an example of a tip region having a trocar tip and a conductive spacer with the same circumference as electrodes. FIG. 9D illustrates an example of a tip region having a conical tip and a conductive spacer with the same circumference as electrodes. FIG. 9E illustrates an example of a tip region configured as a trocar tip, having a spacer with a circumference that is greater than the OD of the electrodes, in which the electrodes include fillets. FIG. 9F shows an example of a fillet that may be used in various examples of the applicators of the present disclosure in combination with other features.

FIGS. 10A-10D show examples of tip regions with three electrodes as described herein. FIG. 10A illustrates an example of the tip region with three electrodes and FIG. 10B is a cross-sectional view of the tip region of FIG. 10A. FIG. 10C illustrates another example of a tip region with three electrodes. FIG. 10D is a cross-section view of the tip region of FIG. 10C.

### DETAILED DESCRIPTION

Described herein are apparatuses and methods for delivering electric treatment to various anatomical structures of a subject (human or animal). While these apparatuses may be especially useful when applying high voltage, pulsed electric fields (e.g., nanosecond pulsed electric fields), the apparatuses and methods described herein may also or alternatively be used with other energy modalities, such as RF energy, microsecond or picosecond pulses, etc. The apparatuses and methods described herein can be used to treat lesions, tumors, nodules and other growth, diseases and conditions, for example, in a target tissue, including various anatomical structures. Such target tissue may include tissue of various anatomical structures accessible via needle penetration through the skin and/or other percutaneous access applications. For example, apparatuses, systems and methods of the present disclosure may apply nanosecond pulsed electrical fields to treat a target tissue to treat lesions, tumors, nodules and other growths on or within various organs, including muscular organs (e.g., smooth muscle, cardiac and skeletal muscle), circulatory organs (e.g., heart, arteries, veins), respiratory organs (e.g., lungs), abdomen and digestive organs (e.g., stomach, duodenum, intestine, liver), urinary organs, (e.g., kidney, ureter, bladder), immune system organs (e.g., lymph nodes, bone marrow, thymus), nervous system organs (e.g., brain, spinal cord, nerve), endocrine organs (e.g., pituitary gland, thyroid, adrenal glands), reproductive organs (e.g., penis, vagina, prostate, uterus, testicle), and/or skeletal organs (e.g., bones).

One example of the use of the apparatuses and methods of the present disclosure is for treatment of the thyroid nodule or thyroid lesions. Benign thyroid nodules are a form of non-malignant hyperplasia of the thyroid gland. They can present a cosmetic nuisance (an unsightly bulge in the neck), and in more severe cases can interfere with swallowing or breathing, cause pain and pressure. In these cases, partial or complete surgical thyroidectomy may be performed, potentially resulting in hypothyroidism, hypoparathyroidism, nerve damage leading to voice impairment and visible scarring. Non-surgical options like radiofrequency (RF) ablation have been used but carry a risk of collateral damage to the recurrent laryngeal nerve, blood vessels and other critical structures. Non-thermal electric treatment, such as the use of nanosecond pulsed electric fields to disrupt cellular organelles, to induce apoptotic-like regulated cell death (RCD), without causing collateral damage to noncellular collagen-rich tissues, nerves and vessels. Nanosecond pulsed electric fields treatment is non-thermal and in combination with various features of the present disclosure provides substantial advantages to the existing surgical excision or thermal ablation, such as from RF ablation. For example, in addition to reducing the risk of collateral damage as stated above, the use of nanosecond pulsed electric fields may also eliminate or substantially reduce scarring or fibrosis and may cause minimal post-procedural pain.

While various following examples are described in reference to treatment of thyroid, however, it shall be understood that such reference is just a non-limiting example for convenience of description and the devices and methods of the present disclosure apply and are intended for use in any target tissue and anatomical structures that allow for percutaneous access.

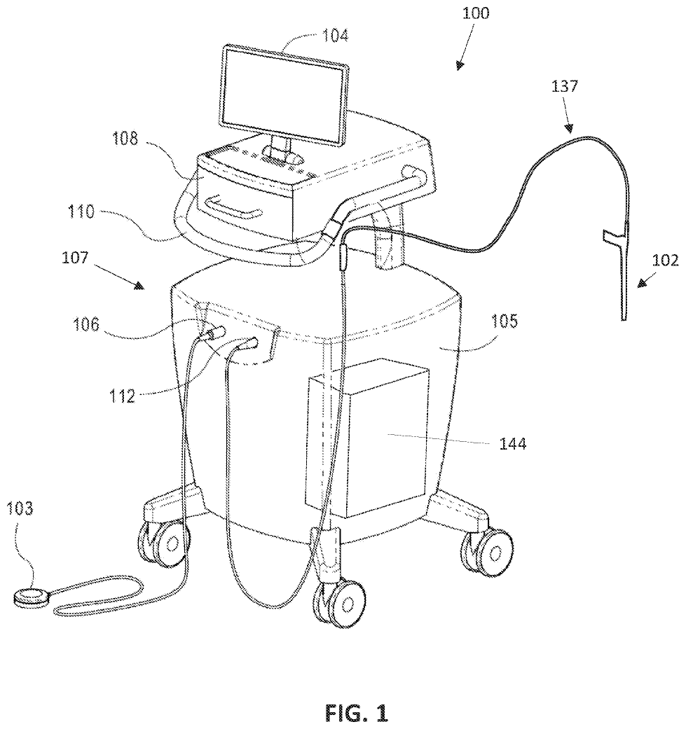

FIG. 1 illustrates one example of a system 100 for delivering high voltage, fast pulses of electrical energy that may include a treatment applicator/tool or apparatus 102 for delivering a pulsed electric field (e.g., sub-microsecond electric field), a pulse generator 107, footswitch 103, and user interface 104. Footswitch 103 is connected to housing 105 (which may enclose the electronic components) through a cable and connector 106. The apparatus 102 may include electrodes and is connected to housing 105 and the electronic components therein through a cable 137 and high voltage connector 112. The system 100 may also include a storage drawer 108 and a console handle 110. The system 100 may also include a holder (e.g., holster, carrier, etc.) (not shown) which may be configured to hold the tool 102. Examples of appropriate applicator tools 102 are described in greater detail below.

In some cases, the applicator tool 102 may include imaging, such as one or more cameras and/or fiber optics, for example, at or near the tip region of the tool. The camera(s) may be forward-facing and/or side facing. The system 100 may be configured to display images (in real time, and/or recorded) in order to identify the target region(s).

A human operator may select a number of pulses, amplitude, pulse duration, and frequency information, for example by inputting such parameters into a numeric keypad or a touch screen of interface 104. In some embodiments, the pulse width can be varied. The system 100 may include a controller 144 (shown schematically in FIG. 1), which may send signals to pulse control elements within system 100 or otherwise control operation of the pulse generator. The controller 144 may include one or more processors and may be coupled to the pulse generator either directly or indirectly. The controller may receive inputs from the one or more inputs and may provide output to the one or more outputs (e.g., monitors/touchscreens/interface 104, etc.). The controller may be a microcontroller. The controller may include control circuitry and may include or be coupled with a memory, communications (e.g., wireless and/or wired) circuitry, etc. The controller may be configured to coordinate the application of energy to the patient. In some embodiments, fiber optic cables are used which allow control signaling while also electrically isolating the contents of the metal cabinet with pulse generation system, e.g., the high voltage circuit, from the outside System 100 may be battery powered instead of being powered from a wall outlet.

The tool 102 may be hand-held (e.g., by a user) or it can be affixed to a movable arm of a robotic apparatus, and its operation may be at least partially automated or fully automated, including computer controlled.

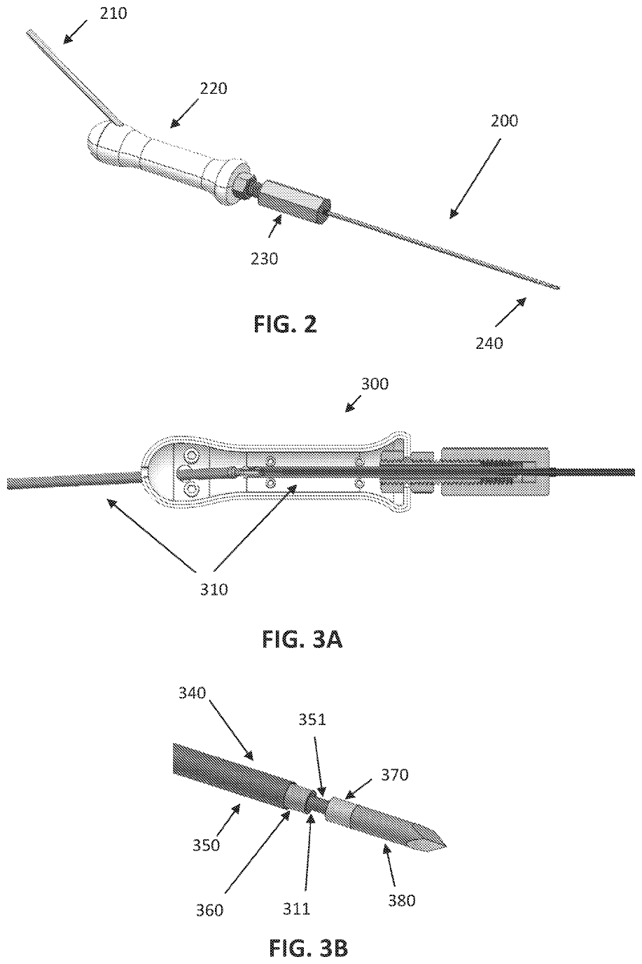

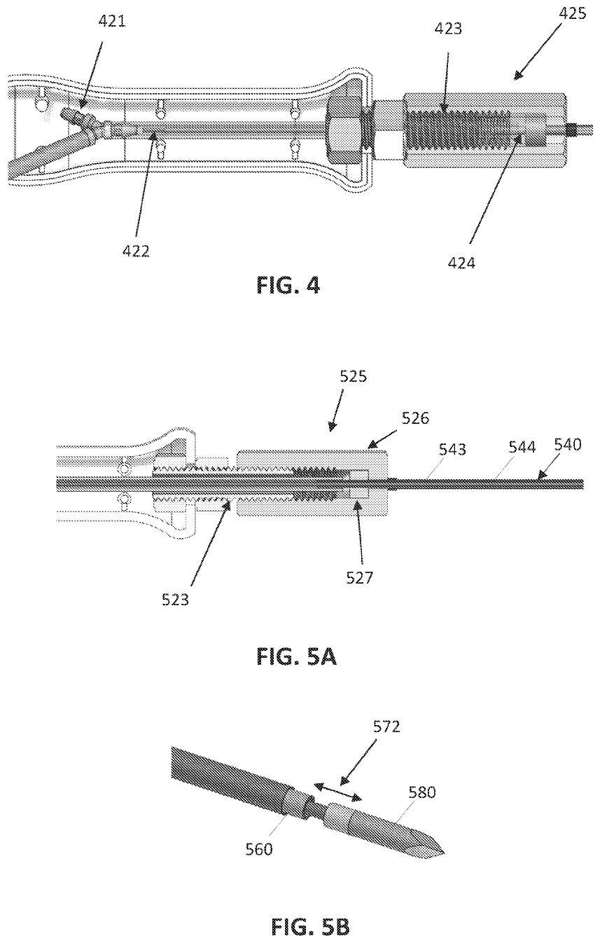

FIG. 2 illustrates the perspective view of an example of a treatment tool/apparatus 200 (which can be used with the system 100 of FIG. 1) as described herein. In this example, the tool 200 includes a vacuum channel and/or an infusion channel 210 (e.g., vacuum line), a handle 220, a tip region 240 and a length adjustment 230 (length adjuster) operably coupled to the handle 220 and to the tip region 240. The length adjuster 230 is configured to adjust a distance between a first electrode and a second electrode as described in more detail in reference to FIGS. 4 and 5A-5C. In the example of FIG. 2, the treatment tool 200 also incorporates a vacuum assist feature including the vacuum channel 210, which allows the apparatus to evacuate extra air from around the electrodes, which in turn reduces the likelihood of arcing between the electrodes. The vacuum channel 210 may be connected to the source of vacuum or suction, for example, a pump or a self-contained vacuum source within the tool 200 itself, for example, within the handle 220. The treatment tool 200 may be connected to a pulse generator (e.g., system 100 of FIG. 1) via electrical cable (not shown). The treatment tool 200 may be connected to the pulse generator via electrical cable. The electrical cable may be electrically isolated (and insulated) from the tool 200, and therefore the operator's hand(s) in the hand-held implementations, by one or more isolation elements. In some examples the handle 220 may be a plastic or insulated housing for the user to hold and apply the pulses to the load. It should be understood, however, that the term “handle”, as used herein, is intended to describe a proximal portion of the treatment applicator and is not limiting. It refers to any structure to support, hold or attach to the treatment tip region with the electrode portion of the device, whether it is intended to be hand-held, or attached to the robotic arm, or for percutaneous or other minimally invasive applications. In some examples the handle may be configured to be hand-held and may include a manual grip. In some examples, the handle may be configured to be held by a robotic manipulator (e.g., arm, etc.).

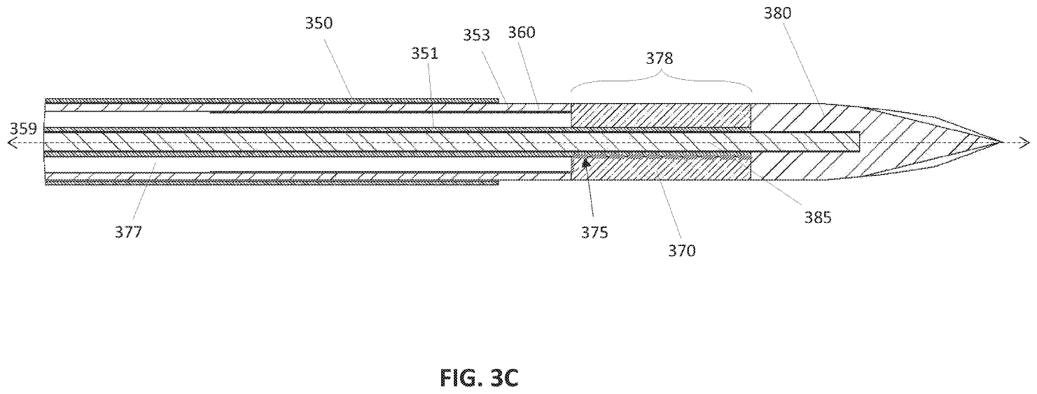

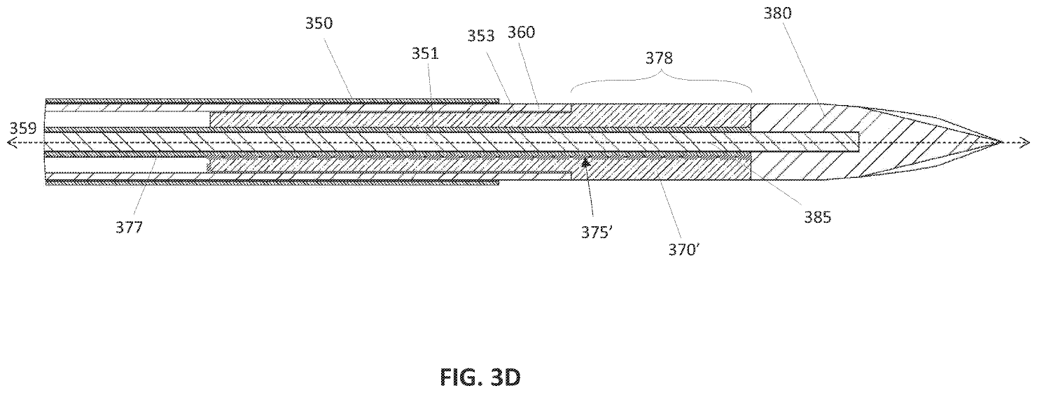

FIGS. 3A-3B illustrate in more detail some elements, including a vacuum-assist feature and an infusion output, that may be incorporated into any of the apparatuses described herein. FIG. 3A shows an example of a section through a treatment tool 300 having vacuum channel 310, which may alternatively or additionally be an infusion channel 310. The vacuum and/or infusion channel 310 may be disposed at least partially inside the handle. The application of vacuum and/or infusion may be controlled via an external vacuum pump (not shown in the drawing). The vacuum channel and vacuum outlet may be used for suction/vacuum to remove any potential air gap between the electrodes. In some examples, the same channel (e.g., tubing) 310 may be used to deliver a saline solution (or multiple different types, e.g., concentrations, of saline solution), which can be used with a vacuum or by itself. In some examples, sterile saline may be infused and could fill the gaps with a conductive solution providing an extra electrical pathway which could potentially enlarge the actual treatment size. In any of these apparatuses, both a vacuum/suction and an infusion channel may be included, so that suction and infusion may be applied concurrently.

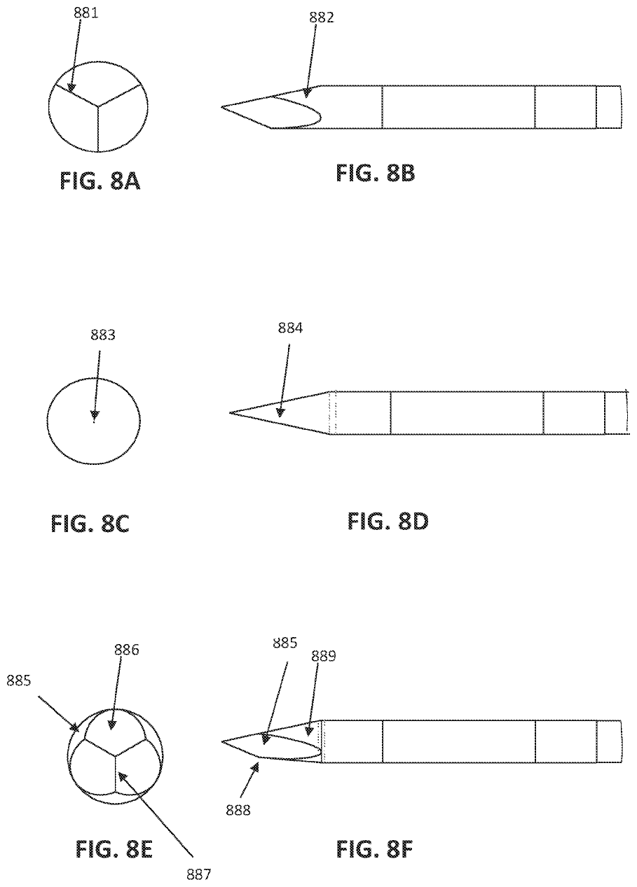

FIG. 3B shows a perspective view of the tip region 340 of the treatment tool 300, which comprises a first (e.g. proximal) electrode 360, a second (e.g., distal or tip) electrode 380, an exterior insulation 350 and an inner insulation 351. While in this example the treatment tool is configured for bipolar application, it should be understood that it can be used also for monopolar energy application. The first electrode and the second electrode may comprise a conductive material, such as a conductive metal (e.g., a stainless-steel material). Since the apparatus of the present disclosure is configured for percutaneous insertion, the tip region 340 may be configured to penetrate the skin and be advanced into the target area for treatment. For example, the second electrode 380, which is at a distal end or forms a distal end of the tip region 340, may be a tissue-penetrating electrode in the form of a bevel, a cone, a trocar (such as 3-sided trocar), or a hybrid tip, depending on requirements. The second electrode (in this example, the distal electrode) may be longer than the first electrode (in this example, a proximal electrode). Other examples of electrodes at the distal end of the tip region (which may be also referred to as the distal electrode) are described in more detail in reference to FIGS. 8A-8F, below.

The exterior insulation 350 and the inner insulation 351 may include, for example, a polymeric insulator, such as a polyimide material. The vacuum and/or infusion channel 310 shown in FIG. 3A may connect to a vacuum outlet and/or infusion outlet 311 as shown in FIG. 3B. In FIG. 3B, the outlet 311 may be disposed between the first electrode 360 and an inner insulation 351. Each electrode may be connected to a high voltage wire (not shown) within the handle, which may connect back to the pulse generator, such as the one shown in FIG. 1. For example, a first wire may connect to the first electrode 360 and a second wire may connect to the second electrode 380. The exterior insulation 350 may insulate the first wire and the inner insulation 351 may insulate the second wire. The electrical lines connecting to the first and/or second electrode may be wires, cylinders, cables, meshes, etc. For example, the electrical line connecting the first electrode 360 to the pulse generator may be coaxially arranged relative to the electrical line connecting the second electrode 380 to the pulse generator. Each pole (e.g., the first electrode and the second electrode) may be electrically isolated. The isolation may be improved by separating and at least partially insulating the poles with a spacer 370 (e.g., a polyimide insulative spacer) that may be disposed between the first electrode 360 and the second electrode 380 and may be configured to electrically isolate the first electrode 360 and the second electrode 380 from each other. The target region can be treated by electrical energy flowing in a bipolar manner between the first electrode 360 and the second electrode 380.

As mentioned, any of the apparatuses described herein may be configured to prevent or reduce arcing, including in particular, arcing between the first (e.g., proximal) and second (e.g., distal) electrodes. In any of the apparatuses described herein the first electrode and the second electrode may be separated from each other by a relatively long minimum clearance (e.g., creepage path) distance. Although the spacers described herein may be generally configured to prevent or reduce arcing, the use of adhesive (e.g., glue) to bond and/or form the spacer may result in entrapment of air bubbles that may in turn lead to arcing. Thus, in any of these apparatuses the spacer may be formed between the first and second electrodes without the use of an adhesive.

Further, in any of these apparatuses the spacer between the first electrode and the second electrode may be configured to maximize or increase the standoff distance (e.g., the minimum clearance or electrical creepage distance). For example, FIG. 3C shows an example of a device including a spacer 370 that is positioned between the proximal electrode 360 and the distal electrode 380. The proximal end of the spacer in this example extends only to the distal end of the proximal electrode. FIG. 3D shows another example in which the spacer 370′ extends further proximally, and in this example extends proximal to the proximal electrode 360. Spacers that extend proximally of the proximal electrode may be particularly advantageous as they may increase the minimum clearance or electrical creepage distance 375, 375″ between the electrodes. In FIG. 3C the minimum clearance or electrical creepage distance 375 extends along the spacer and is approximately the distance between the electrodes 378 (as measured from the outside of the device). In contrast, the example shown in FIG. 3D illustrates a spacer 370′ that extends proximally past the proximal electrode, so that the minimum clearance or electrical creepage distance 375′ is more than twice the minimum clearance or electrical creepage distance in the example shown in FIG. 3C.

Thus, in any of these apparatuses the first electrode may be separated from the second electrode by a spacer that extends between the first electrode and the second electrode, and may extend past one or both of these electrodes, so that a minimum electrical clearance (electrical creepage) distance between the electrodes is greater than the distance separating the first and second electrodes (e.g., 1.5× or greater, 1.75× or greater, 2× or greater, 2.25× or greater, 2.5× or greater, 2.75× or greater, 3× or greater, 3.25× or greater, 3.5× or greater, etc.), for example, when measured from the outside of the apparatus. In some examples, as shown in FIG. 3D, the spacer 370′ may extend proximally of the proximal electrode 360, to the proximal end of the distal electrode 380, which may increase the standoff distance without having to increase the distance between the electrodes.

In both FIGS. 3C and 3D the apparatus includes a proximal electrode 360. The proximal electrode may be formed as a ring extending partially or completely around the outer surface of the device. The proximal electrode may have an insulated inner surface 353. The proximal electrode 360 may be formed of a cylindrical, electrically conductive material that is arranged coaxially over the internal wire 385 or other electrical connection to the distal electrode 380 and over the spacer 370. A portion of the cylindrical, electrically conductive material that does not form the electrode (e.g., the region proximal to the proximal electrode) may be insulated 350, as shown in FIGS. 3C and 3D. The distal end of the apparatus is configured as a tissue-penetrating distal electrode 380 that is electrically coupled to the pulse generator via the internal wire 385 or other electrical connection. In the sectional view shown in FIGS. 3C and 3D the apparatus (or at least the distal end of the apparatus shown) is radially symmetric about the long axis 359 of the apparatus. In this example, the insulated outer surface or outer insulator 350 may be a thin layer adjacent to the proximal electrode (e.g., at the top and the bottom). As mentioned above, the inner surface 353 of the first (proximal) electrode 360 (e.g., the radially inward surface) is also insulated. In addition, the electrical conductor (e.g., wire) connecting the distal electrode 380 to the pulse generator may be insulated by an inner insulator 351, e.g., between the spacer 370, 370′ and the central wire 385 leading to the distal electrode 380. In the example device of FIG. 3D the length of the minimum clearance distance/minimum creepage distance 375″ is greater than 2.75 times (e.g., greater than about 3×) the external distance 378 (actual distance between the distal end of the proximal electrode 360 and the proximal end of the distal electrode 380). In any of the apparatuses described herein the more proximal end region 377 between the outer (e.g., electrode 360) portion and the inner (e.g., wire connector 385) portion may be open; the corresponding distal end region is taken up by the spacer. In some examples this region 377 may be filled with a material, including in some examples a compressible material.

Furthermore, in the example apparatus shown in FIG. 3D, the spacer 370″ is held within the apparatus in a fixed position without the need to use a glue bond or any other adhesive, since the length of the spacer underlying and extending proximally of the proximal electrode may be sufficiently long enough to avoid arcing without the need for a glue bond as an insulator. The spacer may be either insulative or conductive in different embodiments.

As mentioned, the spacer between the electrodes may be insulating or in some examples it may be a conductive spacer. In any of these examples the spacer may be a balloon that may be filled with an insulating or a conductive material. In some examples the spacer may be filled with air.

As mentioned above, any of the apparatuses/treatment tools described herein may be configured to include suction (e.g., vacuum) to assist in holding the electrodes to the tissue to be treated as well as reducing arcing. The vacuum (suction) may pull the tissue onto the electrodes (e.g., the first electrode) and/or may maintain contact with the electrodes. The use of a vacuum may remove or reduce air gaps between the electrodes and the tissue, which may reduce arcing and otherwise improve contact with the tissue. In some variations the suction may be automatically or manually applied before activating the application of the high voltage, fast pulsed electrical energy. Suction may be applied to a predetermined level to prevent damage to the tissue. Once the energy has been applied, the suction may be released, automatically or manually. However, it should be understood that the examples and implementations described herein may be used without the vacuum.

FIGS. 4 and 5A-5C demonstrate an example of a length adjustment feature (length adjuster) that may be present on any of the treatment tools described herein. FIG. 4 shows an example of a handle and length adjuster 425 of the treatment applicator according to the present disclosure. As described in reference to FIGS. 3A-3B, the first and the second electrodes may be each connected to a respective high-voltage (HV) wire. The HV wire allows delivery of the high-voltage electrical field and may be formed of any appropriate material. In the example apparatus shown in FIG. 4, the first wire 422 is connected to the first electrode (not shown) and disposed within the handle and the length adjuster 425. The first wire may be insulated with the exterior insulation (as stated in reference to FIG. 3B) and routed, for example, through a threaded rod 423. A second wire 424 may be connected to the second electrode and may be insulated with the inner insulation (not shown) as stated in reference to FIG. 3B. The second wire 424 may be introduced through a luer-type standard fitting 421 and it may be sealed off for the vacuum or the infusion. In some examples the second wire 424 maybe slidably disposed within the device, including within the elongate shaft 540 (shown in FIG. 5A), so that as the spacing between the electrodes is increased or decreased (e.g., by actuating the length adjuster 425).

In some examples, the wire (electrical connector) includes a region formed of a hypotube (e.g., stainless-steel or other conductive material) which may be full hard, welded and drawn to tight tolerances and which is an effective component for applications requiring strength, uniformity, and corrosion resistance. It can be a high-performance alloy for use in non-implantable medical devices. For example, the wire may include a minimum of 18% chromium and 8% nickel with a maximum of 0.08% carbon. It can be in the chromium-nickel austenitic alloy family.

In the examples shown in FIGS. 2, 3A-3B, 4 and 5A-5B, the length adjuster(s) shown are configured as a threaded member coupled to an extendable (e.g., telescoping) member that may rotate to longitudinally move a first elongate member, to which either the first or second electrode is attached, relative to a second elongate member, to which the other electrode (e.g., the second or first electrode) is attached. The first elongate member may be concentrically arranged relative to the second elongate member. Rotational movement of the length adjuster results in longitudinal movement of the first elongate member relative to the second elongate member, and therefore a change in the spacing between the first electrode and the second electrode at the distal ends of the first elongate member and the second elongate member. The length adjuster may be calibrated (and labeled) so that a specified rotational movement may result in a specified longitudinal movement and, therefore, a specified increase or decrease in spacing (depending on the direction of rotation). In some examples the length adjuster may be configured so that rotation of the length adjuster does not rotate the first or second elongate member. The first and second elongate members may be held within or may form a part of the elongate shaft 540 extending from the handle in a proximal-to-distal direction. The tip region (including the first and second, or more, electrodes) may extend distally from the tip region. The distal tip (and the electrodes thereon) may be formed at the distal end of the shaft or may be coupled to the elongate shaft.

FIG. 5A shows one example of adjusting a treatment length, i.e., adjusting a distance between the first electrode and the second electrode. The length adjuster 525 in this example includes a threaded rod 523 and an adjuster knob 526 disposed on the threaded rod 523, which may allow for the apparatus to adjust spacing between the electrodes, for example, to treat different treatment lengths. As the adjuster knob 526 is rotated clockwise and/or counterclockwise, it pushes against a distance adjustment stator 527, which is attached to the first electrode through an elongate member 543, thereby advancing or retracting the (inner) elongate member 543, and moving the first electrode in or out, which in turn increases or decreases the spacing between each electrode, as illustrated in FIG. 5B. In this example, the second, tissue-penetrating distal electrode 580 is rigidly coupled to the inner elongate member 543, while the first, more proximal, electrode 560 is rigidly coupled to the outer elongate member 544. The example shown in FIG. 5A is non-limiting example, and other length adjustment mechanisms may be used.

For example, a length adjustment mechanism may be configured to longitudinally slide the inner elongate member relative to the outer elongate member by driving a slider on the handle proximally or distally. In some examples the handle may include a gear or gearing for controlling the relative longitudinal movement of the inner and outer elongate members. In some examples a hydraulic or pneumatic mechanism may be used to drive the separation or contraction of the first and second electrodes, e.g., by controlling longitudinal movement of the inner and outer elongate members to which the first and second electrodes are coupled.

In use, the operator may adjust or direct adjustment of the distance between the first electrode and the second electrode depending on the size of the target region so that the electric field can pass through the target tissue properly and efficiently. As mentioned, FIG. 5B shows an example of the tip region of an apparatus with the adjustable treatment length between the electrodes. The spacing between the first 560 and second 580 electrodes may be adjusted as shown by arrow 572. The spacing between the electrodes may be, for example, from between about 1 mm to 7 mm, 1 mm to 6 mm, 2 mm to 7 mm, 2 mm to 5 mm, etc., depending on the application and the energy required. This distance may be adjustable according to the size of the target region. In some examples the region between the electrodes may include a spacer, as mentioned above. The spacer may be configured to expand and contract as the spacing between the electrodes increases or decreases. For example, the spacer may be configured to stretch or compress.

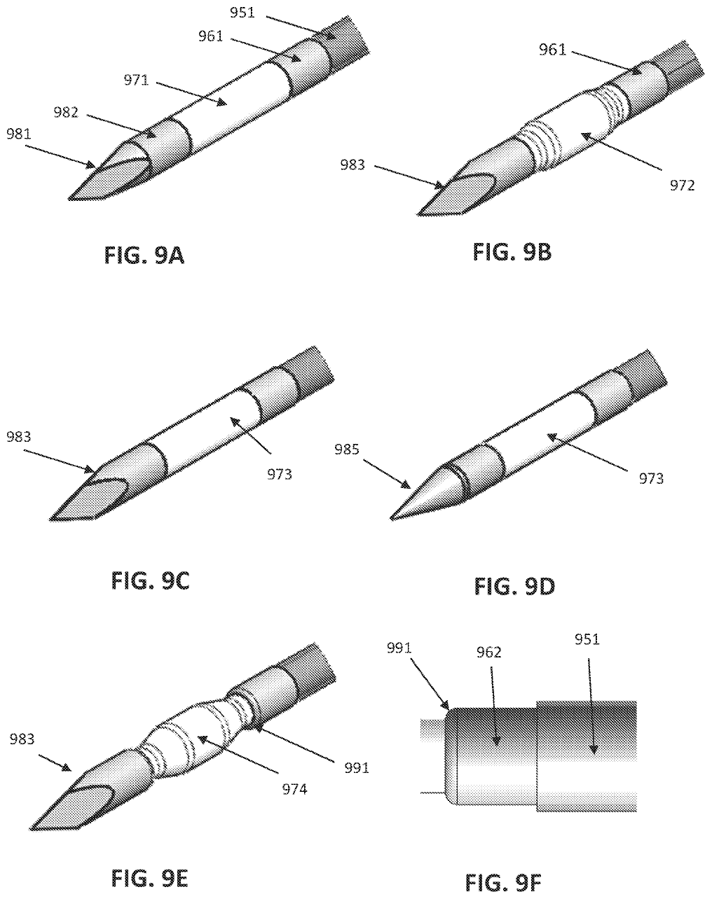

For example, in any of the apparatuses described herein the spacer may be configured to radially expand and/or contract from a flush configuration (see, e.g., the spacer of FIG. 9A, described below) to a more radially expanded configuration (see, e.g., FIGS. 9B and E). Alternatively in some examples the spacer may be configured to retract to have a radial diameter that is equal or less than the outer diameter of the rest of the apparatus (e.g., the proximal electrode). Radial expansion of the spacer may allow the device to create a more intimate contact with the tissue, for example, after insertion and during application of the electric energy. For example, the apparatus may be adjusted to decrease the circumference (e.g., diameter) of the spacer, e.g., by pushing the inner member, for example, by pushing an electrical line or wire coupled to the distal electrode, or to a support coupled thereto, distally. In this example, releasing the force previously applied to push the inner member distally may permit the spacer to revert to expand radially outwards, and/or force may be applied (e.g., by pulling the inner member or wire proximally) to drive the spacer outwards to expand its circumference. Alternatively, in some configurations the apparatus may be configured so that in the neutral configuration the spacer has a circumference/diameter that is relatively small, or smaller than the circumference/diameter of the spacer when force is applied, e.g., pulling the inner member (distal electrode) proximally relative to the outer member (e.g., proximal electrode). Thus, the apparatus may be configured to have a relatively narrow (low) spacer profile for inserting into (and/or removing from) tissue, while the spacer profile may be expanded (or allowed to expand) once in the tissue. This configuration may enhance the ease of inserting/removing the apparatus by changing the outer diameter of the spacer.

For example, the distal electrode may be translated distally and/or proximally to the proximal electrode to adjust the circumference (diameter) of the spacer. The spacer may be any of the spacers described herein, including spacers that are formed of an elastomeric material that may radially expand when compressed longitudinally and may radially collapse when pushed or stretched longitudinally. Thus, the apparatus may be inserted with a constant or relatively low radial size while the spacer (which may be, e.g., an insulating spacer) between the electrodes is the same diameter as the electrodes, but once the electrodes are in place the relative spacing between the proximal and distal electrodes may be adjusted to compress the spacer, so causing it to bulge outward which may create an intimate contact with the tissue.

One or more spacers may be used. In some examples the spacer may be formed of a conductive material or alternatively, an insulating material, such as a polymeric material, and may be attached to either or both the inner and outer elongate members. Alternatively in some examples the spacer may remain a relatively constant size (e.g., length and outer or inner diameter, as shown in FIG. 5B.

Any of these apparatuses may include a lock or securing mechanism to lock or hold the spacing between the first and second electrodes. In some examples the lock may be coupled to the length adjuster. In some examples the lock may be on the handle. The lock may be coupled to the first (e.g., outer) and/or second (e.g., outer) elongate members. The lock may prevent actuation of the length adjuster.

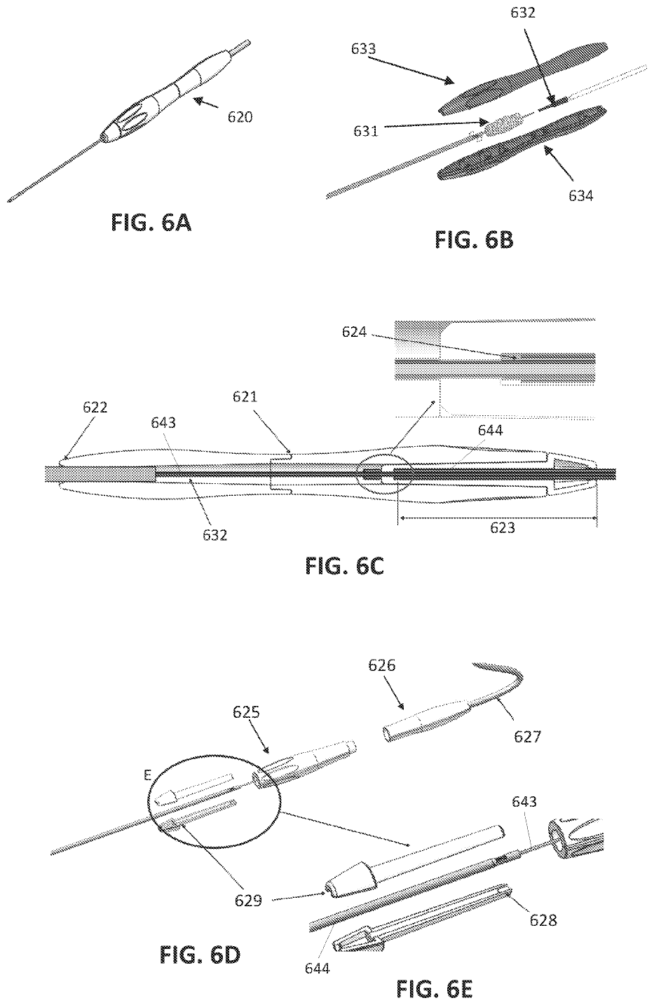

Various designs of the handle may be used in the percutaneous treatment tools described herein. For example, the handle may have a cylindrical or a pencil shape. FIGS. 6A-6E show examples of handles for treatment tools as described herein. In some examples the handle may be a pencil-style handle 620, as shown in FIG. 6A. In general, the handle may act as an interface between the connection to the pulse generator (e.g., FIG. 1) and the electrodes, and may be configured to securely transition between the cabling input from the pulse generator and the applicator (e.g., treatment tool) distal end which interfaces with the target tissue. In particular, the handle may be configured to couple the electrodes at the distal end region of the treatment tool with the high-voltage electrical input from the pulse generator in a manner that prevents harm or risk of shock to the user who may hold or operate the treatment tool by manipulating the handle. Connections between the cabling to/from the pulse generator and the electrodes at the distal end region of the treatment tool may include electrical insulation and/or isolation, including reducing or eliminating insulation creepage, which may be particularly important when using relatively high voltage, sub-microsecond pulses. In general, the handle 620 may be formed of an insulating material (e.g., a polymetric material) that may be configured to secure the cable and the internal electrical connectors to maintain internal and external creepage distances to prevent or reduce the risk of harm to the user. For example, in FIG. 6A, the handle is substantially hollow and includes internal channels and/or ribs to secure an end of the cable to/from the pulse generator and/or the inner elongate member and outer elongate members which may be coupled to and/or include the first and second wires for making electrical connections between the pulse generator and the electrodes, as mentioned above. The internal structure of the handle may include one or more insulating baffles to increase the creepage distance, e.g., between the inner elongate member (forming, coupled to or enclosing the second wire) and the outer elongate member (forming, coupled to or enclosing the first wire).

The handle may be reusable or disposable. As shown in FIG. 6B, in some examples the handle may comprise a top handle half 633 and a bottom handle half 634 configured to permanently or releasably couple together. As shown in the exploded view of FIG. 6B, the handle may also comprise a baffle 631 and an insulated connection 632 to the cabling to connect to the pulse generator. In FIG. 6B this insulated connection 632 is a heat-shrink connection which may act as a barrier to help achieve additional creepage length and clearance distances. Baffle 631 may be configured to increase a minimum clearance distance between the conductive parts that may further reduce risk of current leaking which may otherwise damage the apparatus and/or risk harm to the user. The minimum clearance distance, as used in the present disclosure, may indicate the shortest distance that avoids current leakage in the air or along an insulating material surface path. In other words, the minimum clearance distance can include a distance that is the greater of the following two distances: 1) a shortest distance or path that prevents current leakage between two conductive parts measured along any surface or combination of surfaces of an insulating material, and 2) the shortest path in air between two conductive parts that prevents current leakage. A “creepage distance” includes a shortest distance that prevents current leakage (e.g., in some examples, arcing) along the surface of the insulating material between two conductive parts, as defined by the International Electrotechnical Commission (IEC), or as otherwise known in the art. It can include the surface distance from one conductive part to another conductive part, or an area accessible by a user. “Air clearance” includes the shortest path that prevents arc in air between two conductive parts as defined by the IEC, or as otherwise known in the art. It can include the uninterrupted distance through the air or free space from one conductive part to another conductive part or an area accessible by a user.

In the example shown in FIG. 6B, the baffle 631 inside the handle 620 increases the minimum clearance distance as the length along the surface of the baffle increases. The baffle 631 can create, for example, 20 mm to 80 mm of the minimum clearance distance, 30 mm to 70 mm of the minimum clearance distance, 40 mm to 60 mm of the minimum clearance distance, etc. In FIG. 6B, the heat shrink insulation 632 can cover high voltage wire and solder joints to prevent internal creepage. The baffle 631 and similar structures for increasing minimum clearance distance may be formed as described in various examples of the co-owned Patent Publication US 2019/0269904 A1, which is incorporated herein for reference.

FIGS. 6C-6E illustrate another example of a handle for a treatment tool as described herein. In FIG. 6C the handle includes a distal handle section and a proximal handle section that are coupled together, e.g., at a bonding region 621. The proximal end of the handle is secured, e.g., by an adhesive 622, to the cabling configured to couple to the pulse generator. The cabling may include two or more wires that are safely distributed within the handle to the electrodes at the distal end region of the device (not shown). For example, in FIG. 6C, insulating heat shrink material 632 may cover a first high-voltage wire from the cabling that is coupled to the inner elongate member 643. This insulating heat shrink material, as well as solder joints between the wire and the inner elongate member, may prevent internal creepage and clearance issues, as described above. The handle may also include one or more (or a plurality) of internal supports or structures that are configured to secure the elongate members (e.g., the inner 643 and outer 644 elongate members) within the handle and may help isolate and prevent creepage of leak current. The elongate members may be insulated along their length, and this insulation may extend into and within the handle. For example, the outer member or shaft 644 may include outer shaft insulation 624 that extends into the handle to prevent creepage. In addition, the handle may be dimensioned to include additional minimum clearance to reduce current leakage (creep) from the connection between the wires and the elongate members. For example, in FIG. 6C, the spacing 623 between the distal end of the handle and the end of the outer elongate member, where the electrical connection to the first wire may be made, may be greater than 48 mm, so that the external minimum clearance distance is greater than 48 mm.

FIG. 6D shows an exploded view of the handle of FIG. 6C. In this example the proximal handle section 626 is shown separated from the distal handle section 625. As mentioned, the proximal end couples to the cable 627 configured to connect to the pulse generator. In the example shown in FIGS. 6C-6E the handle also includes one or more shaft (elongate member) locators 629 that may help secure, and in some examples electrically isolate, the elongate member(s) within the inside of the handle. In FIG. 6D the shaft locators include an inserted holder 629 that is shown in more detail in FIG. 6E. The shaft locator includes an internal channel in which the shaft(s) 644, 643 of the tool may sit. The shaft locator also includes one or more internal boss 628 that may mate with a seating region (e.g., notch) in the shaft(s) to limit or prevent (e.g., constrain) movement in the longitudinal (axial) direction.

### ARC Mitigation Solutions and Examples

According to another aspect of the present disclosure, any of the apparatuses (e.g., devices, systems, etc.) described herein may be configured to prevent or at least reduce arcing between the electrodes of the apparatus of the present disclosure. As discussed above in reference to FIGS. 2 and 3A-3B, this may be achieved at least in part by using a vacuum to remove air from around (e.g., between) the electrodes or in reference to FIGS. 3C and 3D by coupling the spacer within the distal end region without adhesive. In some examples this can be achieved without the use of vacuum or in addition to the use of vacuum, e.g., by implementing various features, alone or in various combinations, as described herein.

### Reduction Of Peak Electric Field

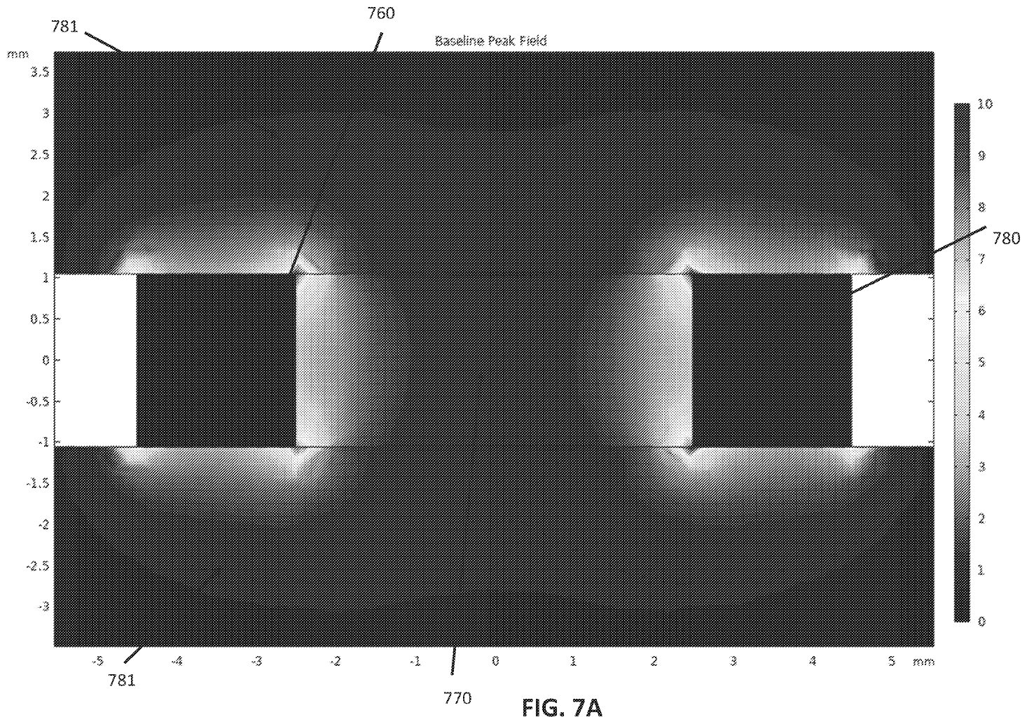

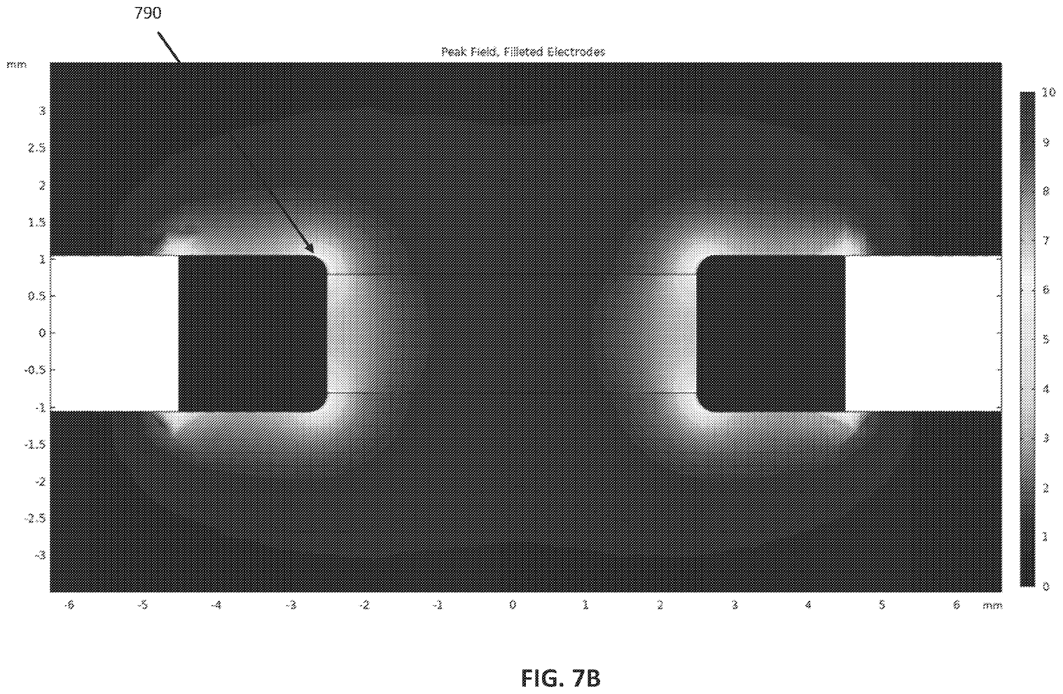

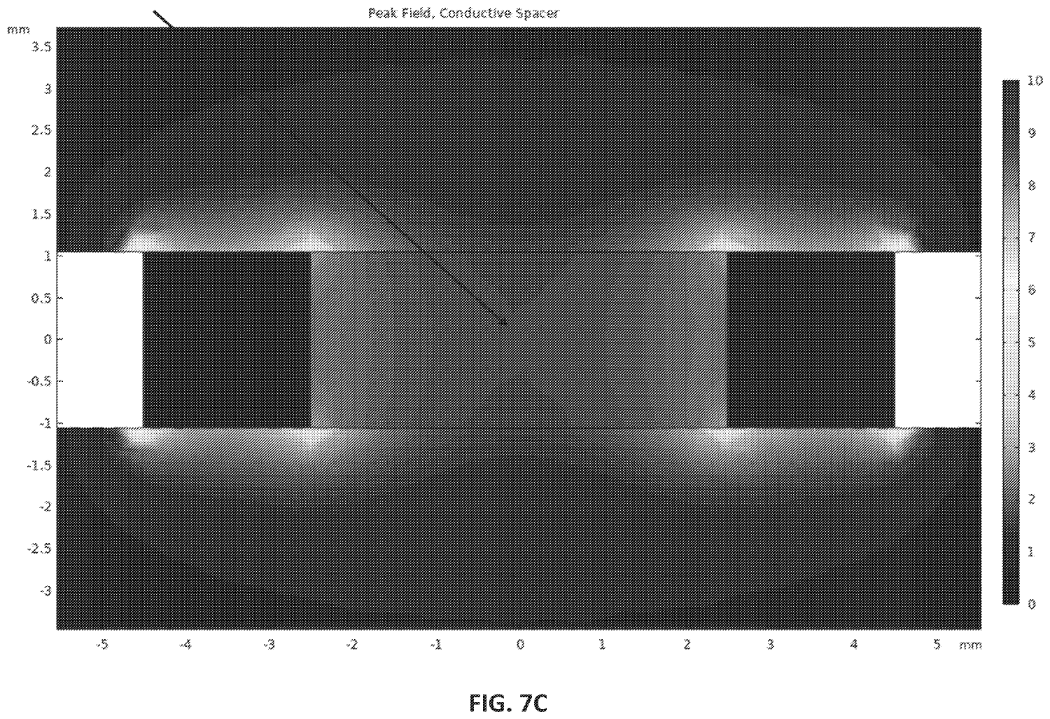

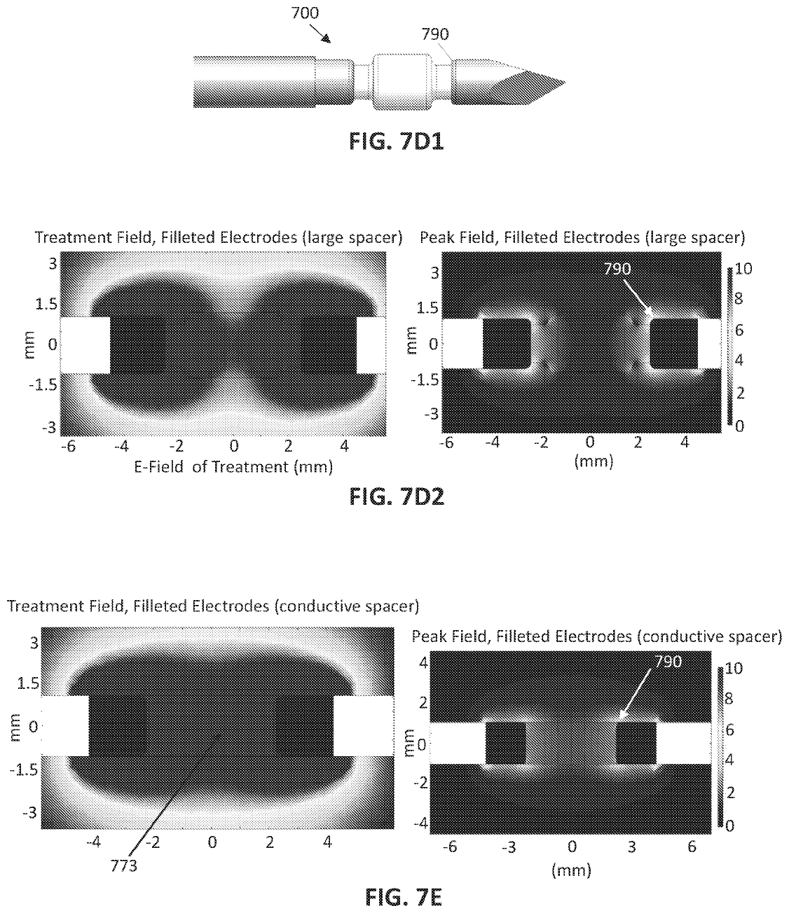

While in various applications it may be important to maintain the high level of the treatment electric field for successful treatment, however, at the same time the peak electric field may be reduced. Typically, a peak electric field may vary depending on the type of edge of the electrodes in the tip region. For example, the peak electric field usually appears near edges of each electrode. It may be helpful to reduce the peak electric filed since the higher peak electric fields are more likely to arc between electrodes. The electrode design can cause the peak electric fields to be different for the same voltage potential. Reducing the peak fields for a given potential can reduce the risk of arcing. The electric fields can be concentrated by sharp edges of the electrode and abrupt changes in conductivity. Therefore, according to some examples of the present disclosure peak electric field can be reduced by rounding corners, e.g., edges, of the electrodes (providing, for example, fillets) as described in reference to of FIGS. 7B and 7D below. Another feature that allows to reduce peak electric field according to the present disclosure is providing a conductive spacer as described in reference to FIGS. 7C and 7E below. Additional feature that allows to mitigate arcing between the electrodes according to the present disclosure comprises provided a spacer (either insulative or conductive) that has at least a portion with a circumference or diameter that is larger than a circumference or a diameter of the electrodes. Such feature is demonstrated in FIG. 7D1.

According to one example of using the devices and methods of the present disclosure, nanosecond pulsed electric treatment was performed on the thyroid glands of four Yorkshire pigs using a treatment tool as described herein, having an electrode array inserted through a small incision in the neck and into one side of the thyroid lobe. Histological assessment of the treated tissue was performed at 0, 2, 8, and 30 days post treatment to determine the impact of the treatment on the parenchymal and stromal portions of the gland. Resulting intense Caspase-3 staining throughout the treatment zone at day 0 indicates that the treatment performed can initiate programmed cell death in a spatially defined region. At 30 days, pronounced parenchymal loss was evident within the treatment zone with minimal inflammation, continued phagocytosis and collagen remodeling.

The results show that nanosecond pulsed treatment as described herein may be a useful, minimally invasive technique to treat, for example, benign thyroid nodules while sparing the surrounding normal thyroid tissue and reducing risk of collateral damage to nerves and vessels.

FIGS. 7A-7E show examples of the results of the COMSOL analysis of the electric fields distribution corresponding to the treatment tools with different features configured to reduce peak electric field according to an aspect of the present disclosure. FIG. 7A shows an example of the baseline electric field without curved edges (filets), conductive spacer, or larger-diameter spacer features as described herein. Specifically, in the example of FIG. 7A, the electrodes 760, 780 have a diameter of 2 mm and are separated by an insulative 5 mm long spacer 770. As shown in FIG. 7A, the peak electric field in the thyroid tissue 781 was measured and used as a baseline to compare with the peak electric field using the tool configurations that incorporate various features described herein. As mentioned, the peak electric field appeared at the edges of the electrodes where an abrupt change in the conductivity occurs since the electrodes 760, 780 are in contact with the insulative spacer 770 and the thyroid tissue 781.

FIG. 7B shows an example of the electric fields of the treatment tool having fillets (rounded corners). As seen in FIG. 7B, adding a rounded corner 790 can reduce the peak electric field, for example, by 15% to 30%, by 20% to 25%, depending on the type of the tissue. In this example, with the same spacing of 5 mm between the electrodes and the same electrode outer diameter of 2 mm in the thyroid tissue, the peak electric fields showed about 19% reduction compared to the baseline of FIG. 7A. Any appropriate radius of the curvature for the fillet 790 may be used, for example, if the electrode has a thickness of t, the radius of curvature may be larger than about t/8 (e.g., larger than about t/7, larger than about t/6, larger than about t/5, larger than about t/4, larger than about t/3, between about t/8 and about 4t, between about t/8 and 2t, etc.). In general, a larger radius of curvature may be preferred. For example, in some apparatuses the radius of curvature of the curved edge (fillet) may be, e.g., between about 0.1 mm to 0.5 mm for certain dimensions of the electrodes.

FIG. 7C shown an example of electric fields of the treatment tool having a conductive spacer between the electrodes. In this example, the conductive spacer 771 has the same circumference as the electrodes. Conductive spacer 771 may be made from any suitable conductive material having a desired conductivity. For example, conductive spacer may be made from a hydrogel, a conductive adhesive, a conductive gel, a conductive silicone, a urethane rubber, carbon nanotubes, or any combination thereof. The desired conductivity of the conductive spacer 771 may be selected, for example, based on the conductivity of the tissue being treated or conductivity of the skin/tissue at the percutaneous introduction of the apparatus. For example, the conductivity of the conductive spacer may be substantially the same as conductivity as the treatment area, in some example, up to ten times (10×) the conductivity of the treatment area, and up to approximately one hundred times (100×) the conductivity of the treatment area. In some embodiments, the conductivity of the conductive spacer may vary throughout the conductive spacer 771. For example, the conductive spacer may have zones that have different conductivity and/or there may be a gradient of conductivity within the conductive spacer. In the implementations according to the present disclosure, selecting the electrode assembly may be based on the conductive spacer having a conductivity that is less than, greater than or equal to a conductivity of a tissue of the treatment area. In some examples, the method may include selecting the electrode assembly based at least in part on a conductivity of the conductive spacer. In other examples, selecting the electrode assembly may be further based at least in part on a size of the electrode assembly and a size of the treatment area. In some implementations, the method of treating a tissue with a pulsed electric field may include selecting the voltage to be applied to the treatment area based at least in part on a conductivity of the conductive spacer.

The consistency of the conductive spacer 771 may be solid, compressible, or gelatinous yet firm enough to maintain shape and position within electrode assembly. The conductive spacer 771 alone can reduce the peak electric field by 25%-50% compared to the electrodes with the insulative spacer since the conductive spacer can relieve the abrupt change in the conductivity at the edge of each electrode. Under the same condition as FIG. 7A in the electrode circumference and the spacing between the electrodes, in this example the peak electric field demonstrated about 41% reduction compared to the baseline of FIG. 7A.

FIG. 7D1 illustrates a treatment tool 700 having fillets 790 on the electrodes and an insulative spacer with a circumference larger than the circumference of the electrodes. FIG. 7D2 illustrates the corresponding electric fields. The larger circumference or diameter on at least the portion of the spacer increases tissue contact pressure which, in turn, also mitigates the risk or arcing. Under the same condition as FIG. 7A in the electrode circumference and the spacing between the electrodes in the thyroid tissue, this example shows about 21% reduction in the peak electric field compared to the baseline of FIG. 7A.

FIG. 7E illustrates electric fields corresponding to the treatment tool having a conductive spacer with the same circumference (e.g., OD) as the electrodes. In this example, the peak electric field at electrode/conductive spacer junction due to the combination of the fillets and the conductive spacer is reduced by about 43% compared to the baseline of FIG. 7A. Also, as shown in the left image of FIG. 7E, conductive spacer increases treatment field in the middle between the electrodes, which may provide further advantage of more uniform treatment in the area in the middle between the electrodes.

As mentioned above, any of the treatment tool configurations shown in FIGS. 7A-7E may be implemented with a treatment length adjustment feature as described above. Depending on the particular configuration, any appropriate modifications may be made to the length adjuster as will be understood by those skilled in the art. In some examples when a conductive spacer is used, it may be desirable to use two or more conductive spacers each adjacent to the respective first and second electrodes, and these conductive spacers may be separated by a gap that allows for a length adjustment and sliding in and out.

### Increasing Hoop Stress

Good contact between the tissue to be treated and the material between the electrodes (e.g., a spacer) may be important for are mitigation or prevention. Any air path or fluid path directly from one electrode to another electrode can provide an arc path. Therefore, increasing the hoop stress on the tissue may provide a better seal against the spacer. The hoop stress may be introduced by stretching the tissue rather than cutting the tissue while inserting the tip region of the tool into the target tissue. Some examples of the configurations of the tip region (including the distal electrode) for increasing the hoop stress according to the present disclosure include: (i) increasing circumference (e.g., diameter) of a spacer relative to the circumference (e.g., diameter) of the electrode to force the tissue to stretch in that zone and make a good contact with the electrodes and the space between the electrodes, (ii) decreasing the cutting circumference (e.g., diameter) of the distal end of the tip region to make the cutting section smaller than the electrode circumference/diameter, and/or (iii) using a tip with no cutting edges, where the tip insertion only stretches the tissue Finally, the tip may avoid abrupt changes in the angle of the surface (particularly in otherwise flat surfaces) that may introduce a gap between the tissue and the surface.

FIGS. 8A-8F show examples of the configurations of a distal end of the tip region (distal electrode) according to the present disclosure. The treatment tools of the present disclosure may also be referred to as the percutaneous needle electrode. FIGS. 8A-8B show a front view and a side view, respectively, of an example of a trocar-shaped distal end or tip. The trocar tip 882 penetrates the tissue by cutting the tissue. Three cutting edges 881 can reach to the full circumference of the needle tip, which means that the trocar tip 882 is more effective to cut tissue rather than stretch or expand the tissue. While a trocar tip may not be optimal on its own for the purposes of the present disclosure, it may still be useful when combined with other features of the present disclosure, such as spacers with the increased circumference, conductive spacers, or rounded corners of the electrodes. Further, in some examples (described below) the angles between the faces of the trocar may be configured to minimize the introduction of gaps or spaces with the tissue.

FIGS. 8C-8D show a front view and a side view, respectively, of an example of a conical distal end or tip. The conical tip (“cone” or “pencil”) penetrates the tissue by expanding the tissue rather than by cutting it. As shown in FIGS. 8C-8D, the conical tip 884 does not have the cutting edges (as shown by a smooth surface 883) found in the trocar tip. The conical tip may be effective to stretch or expand the tissue when the tip region with the distal conical electrode is inserted into the target tissue. Therefore, the conical tip may help to make good contact between the tissue and the tip region. However, a relatively higher pressure may be needed when a conical tip is inserted into the tissue since there is no cutting edge in the conical tip.

FIGS. 8E-8F show a front view and a side view, respectively, of an example of a hybrid (e.g., pencil-trocar) distal end or tip. The hybrid tip 889 may be a combination of the cone/pencil tip and the trocar tip, and it is especially useful in obtaining good contact with the tissue and increasing hoop stress. The hybrid tip penetrates the tissue by only initially cutting the tissue to, for example, a circumference or diameter smaller that the full outer circumference or diameter of the tip, which helps with the initial insertion of the tool. Then the hybrid tip only expands the tissue to the full circumference by stretching. The cutting portion of the circumference may extend along the length of the tip from the distal pointed end to a proximal length at which the circumference is about 5% to about 50% of the full circumference of the tip of the electrode (e.g., at the proximal end of the tip) but is not limited to these ranges. As shown in FIG. 8E, the front view the hybrid pencil-trocar tip shows the conical non-cutting section 885 and the trocar style cutting section 886 with 3 cutting edges 887. The cutting edge portion of the tip ends at 888, at about 40% of the length of the tip, at which point the tip has a smaller circumference than the full circumference of the distal electrode. The hybrid tip 889 can be effective to easily penetrate but then stretch or expand the tissue when inserted into the target tissue compared to either the trocar type tip or the cone tip (e.g., conical tip). Due to is cutting edges 887, it requires less pressure than the cone tip when inserted into the tissue, which make it easier to operate.

In general, a hybrid tip as described herein may include a distal cutting portion having one or more cutting edges (e.g., blades), such as a 3-sided trocar as described above, and a larger-circumference (larger outer diameter) smooth, flat or otherwise non-cutting surface that is proximal to the cutting edges. The non-cutting surface that is more proximal may therefor stretch and expand the tissue as the tip is advanced. Although conical (cone) shaped proximal regions may be used, other non-conical shapes may also be used, including other flat or curved surfaces. As described above, shapes that transition gradually during expansion of the tissue so as not to create gaps between the tissue and the electrode tip may be desirable, to prevent arcing.

In any of the tips described herein the tip profile may be configured to prevent abrupt changes in the angle between the wall(s) of the tip and the tissue, which might otherwise introduce gaps between the tip and the tissue that may allow for arcing and/or poor electrical contact. For example, in reference to the hybrid tips described in FIGS. 8E-8F, the transition between the flat (e.g., monotonic) wall(s) forming the trocar region and the conical region may be sufficiently shallow (e.g., change at an angle of about 20 degrees or less, 19 degrees or less, 18 degrees or less, 17 degrees or less, 16 degrees or less, 15 degrees or less, 14 degrees or less, 13 degrees or less, 12 degrees or less, 11 degrees or less, 10 degrees or less, etc.) so that the tissue remains in contact with the tip. If the wall transitions from a less steep region of the trocar to a steeper region of the cone, then the transition may act more like a step and the tissue may lose contact in this region, which could cause corona or arcing between the tissue and the metal electrode tip. Alternatively, in some examples it may be beneficial to include a curved transition between different angled regions of the tip.

FIGS. 9A-9F show various examples of the tip region configurations of the treatment tool of the present disclosure. Various combinations of the novel features of the present disclosure are shown. To increase the hoop stress, a hybrid tip (electrode) or a spacer with a greater circumference than the shaft circumference can be used. FIG. 9A illustrates an example of a tip region having a hybrid tip 982 and a spacer 971 with the same circumference as the shaft and the electrodes 961, 982. The hybrid tip 982 electrode includes a trocar style cutting distal section and a smooth non-cutting proximal section so that the proximal circumference (e.g., diameter) at the cutting edge end 981 is smaller than the full diameter of the shaft. The spacer 971 can be an insulative spacer or a conductive spacer which has the same circumference as the shaft (electrodes). The spacer 971 connects to the first electrode 961 which is proximally insulated by the exterior insulation 951. The tip region in FIG. 9A can increase the hoop stress by the hybrid tip to make good contact between the tissue and the tip region.

FIG. 9B illustrates an example of a tip region having a trocar tip 983 and a spacer 972 with a circumference that is greater, at least along a portion of a length of the spacer, than the circumference of the electrodes 983, 961. The spacer can be an insulative spacer or a conductive spacer which has the greater circumference than the electrodes. The greater circumference of the spacer can force the tissue to stretch in that zone thereby introducing the hoop stress that may aid in mitigating/preventing arcing.

FIG. 9C illustrates an example of a tip region having a trocar tip 983 and a conductive spacer 973 with the same circumference as electrodes. This type of the tip region can decrease the peak electric field by using the conductive spacer 973 as explained in reference to FIG. 7C, above. FIG. 9D illustrates an example of a tip region having a pencil tip 985 and a conductive spacer 973 with the same circumference as electrodes. This type of the tip region can also decrease the peak electric field by introducing the conductive spacer 973 and further can force the tissue to stretch as the pencil tip 985 advances into the tissue, to mitigate/prevent arcing.

FIG. 9E illustrates an example of a tip region having a trocar tip 983 and a spacer 974 with a circumference that is greater (at least along a portion of a length of the spacer) than the circumference of the shaft where the electrodes are positioned; the electrodes include rounded edges (e.g., fillet) 991 at the edge facing the spacer 974. The spacer 974 can be an insulative spacer or a conductive spacer. This type of the tip region can decrease the peak electric field by introducing the fillet 991 as explained in reference to FIG. 7B and further can increase the hoop stress by using the greater circumference of the spacer than the shaft which can force the tissue to stretch in that zone to mitigate and/or prevent arcing. FIG. 9F shows an example of a rounded edge (e.g., fillet) 991 where the edge of the electrode 962 facing toward the spacer 974 is round. The first electrode 962 is insulated by exterior insulation 951.

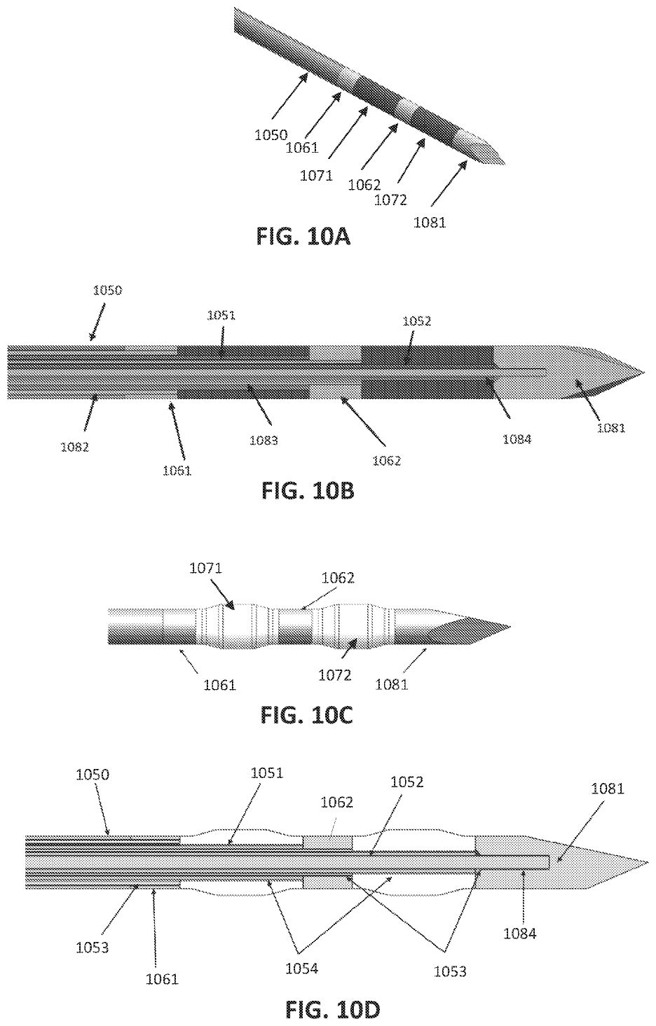

FIGS. 10A-10D show examples of tip regions of a treatment applicator with three electrodes. FIG. 10A illustrates a perspective view of a tip region with three electrodes. FIG. 10B illustrates a cross-sectional view of the tip region with three electrodes shown in FIG. 10A. The electrodes can be bipolar or monopolar. When the tip region is configured to deliver energy in a bipolar manner, a proximal electrode 1061 and a tip electrode 1081 may be positive electrodes and a middle electrode 1062 may be a negative electrode, or the proximal electrode 1061 and the tip electrode 1081 may be the negative electrodes and the middle electrode 1062 may be the positive electrode. Both a proximal spacer 1071 and a tip spacer 1072 can be an insulative spacer or a conductive spacer, or one of the proximal spacer 1071 and the tip spacer 1072 can be the insulative spacer and the other of the proximal spacer 1071 and the tip spacer 1072 can be the conductive spacer. An exterior insulation 1050 can circumferentially insulate the first (outer) hypotube 1082 which connects to the proximal electrode 1061 so that the proximal electrode 1061 is only exposed over the outside region to make contact with the tissue. As shown in FIG. 10B, an inner (e.g., “middle”) insulation region 1051 can insulate the inner surface of the first hypotube 1082 and the outer surface of a second hypotube 1083 from each other. The second hypotube 1083 may connect to the middle electrode 1062 to make electrical contact between the electrode and the pulse generator (and therefore the tissue). A second inner insulation 1052 may insulate the inner surface of the second hypotube 1083 and the outer surface of the third hypotube or rod 1084 (e.g., shown as a rod in FIG. 10B) from each other. The third hypotube or rod 1084 connects to the tip electrode 1081. As mentioned, the tip electrode can be any type of the tissue-penetrating tip (e.g., needle) such as a trocar tip, pencil tip, or hybrid tip. Each of the exterior insulation 1050, the middle insulation 1051 and the inner insulation 1052 can be the same or a different insulator and have the same or different thicknesses. For example, in FIG. 10B, the exterior, and both inner insulation layers may be formed of a polyimide insulation and have a thickness, for example, of between about 0.0005-0.05 inches, 0.001-0.01 inches, or 0.005 inches. The first hypotube 1082 may have a thickness, for example, of between about 0.005-0.5 inches, 0.01-0.1 inches, or 0.065 inches. The second hypotube 1083 may have a thickness, for example, of between about 0.005-0.5 inches, 0.01-0.1 inches, or 0.036 inches. The third hypotube or rod 1084 may have a diameter or thickness, for example, of between about 0.005-0.5 inches, 0.01-0.1 inches, or 0.020 inches. The proximal electrode 1061 and the middle electrode 1062 can be welded to the first hypotube 1082 and the second hypotube 1083, respectively. The apparatus with the three electrodes may enable to treat a larger area of the target tissue.

FIG. 10C shows another example of a tip of a treatment tool including three electrodes: a first (proximal) electrode 1061, a second (middle) electrode 1062, and a third (distal) electrode 1081. The third electrode may be configured as a tissue-penetrating tip electrode as described herein. In FIG. 10C, the tissue-penetrating tip electrode is configured as a trocar. As in the example shown in FIGS. 10A-10B, the first and third electrodes may be electrically coupled to a provide an opposite polarity as compared to the second (middle) electrode. In this example the first, second and third electrodes are separated by spacers (e.g., insulating or conductive spacers) that have a larger circumference than the OD of the adjacent annular electrodes.

FIG. 10D shows a sectional view through the tip of the treatment tool shown in FIG. 10C. The construction of the tip shown in FIG. 10D is similar to that shown in FIG. 10B, and includes an outer insulation 1050 (e.g., in one example, formed of 0.005″ polyimide that is bonded to the outer shaft). The third electrode 1061 is formed of 0.085″ OD stainless steel tubing that is soldered 1053 to the outer hypotube. A first inner insulation layer 1051 (e.g., formed of 0.005″ polyimide in this example) is bonded to the middle shaft, to which the middle electrode 1062 is also soldered 1053. The third, distal electrode (formed as a trocar tip) 1081 is soldered to the inner shaft 1084 (shown in this example as a stainless steel rod having a 0.020″ OD). The inner shaft is also insulated by a second inner insulation layer (e.g., a 0.005″ polyimide insulator bonded to the inner shaft). A first spacer 1054 and a second spacer 1054′ are bonded to the insulation.

According to a further aspect of the present disclosure, methods of treatment of a target tissue are provided. In some examples, the method may comprise inserting percutaneously a tip region of a treatment tool into a target tissue, the tip region comprising a first electrode and a second electrode; and applying a plurality of electrical pulses having a pulse duration of less than 1000 nanoseconds while reducing peak electric field for a given potential and/or increasing hoop stress on the target tissue to prevent or at least reduce arcing between the first electrode and the second electrode. The method may be performed under image guidance, for example ultrasound imaging, or robotic system imaging. The method may comprise navigating and tracking percutaneous insertion of the tip region of the treatment tool, treatment planning and confirmation. In some examples, reducing the peak electric field comprises using a conductive spacer between the first electrode and the second electrode or using electrodes with the rounded corners. In some examples, increasing the hoop stress comprises using a spacer between the first electrode and the second electrode and wherein at least a portion of a circumference or a diameter of the spacer is larger (either permanently or only after being placed within a target area) than a circumference or diameter of each of the first electrode and the second electrode.

As state above, the methods of the present disclosure may be used to treat lesions, tumors, tissue disorders and other abnormalities in or within a muscular organs, circulatory organs, respiratory organs, abdomen and digestive organs, urinary organs, immune system organs, nervous system organs, endocrine organs, reproductive organ, or skeletal organs.

For example, in some implementations, a method of treating a thyroid module is provided. The method comprises inserting percutaneously a tip region of a treatment tool into a target thyroid nodule, the tip region comprising a first electrode and a second electrode; and applying through the first electrode and the second electrode a plurality of electrical pulses having a pulse duration of less than 1000 nanoseconds to thyroid nodule while reducing peak electric field for a given potential and/or increasing hoop stress on the thyroid nodule to prevent or at least reduce arcing between the first electrode and the second electrode. Thyroid nodule may be a benign thyroid nodule. The method may also be conducted under the image guidance.

As mentioned above, any of the apparatuses described herein may be implemented in robotic apparatus that may be used to position and/or control the electrodes during a treatment. For example, a robotic apparatus may include a movable (robotic) arm to which the treatment apparatus or tool is coupled. Various motors and other movement devices may be incorporated to enable fine movements of an operating tip of the apparatus in multiple directions. The robotic apparatus and/or system may further include at least one image acquisition device (and preferably two for stereo vision, or more) which may be mounted in a fixed position or coupled (directly or indirectly) to a robotic arm or other controllable motion device. In some embodiments, the image acquisition device(s) may be incorporated into the apparatus of the present disclosure.

Embodiments of the methods of the present disclosure may be implemented using computer software, firmware or hardware. Various programming languages and operating apparatus may be used to implement the present disclosure. The program that runs the method and apparatus may include a separate program code including a set of instructions for performing a desired operation or may include a plurality of modules that perform such sub-operations of an operation or may be part of a single module of a larger program providing the operation. The modular construction facilitates adding, deleting, updating and/or amending the modules therein and/or features within the modules.

In some embodiments, a user may select a particular method or embodiment of this application, and the processor will run a program or algorithm associated with the selected method. In certain embodiments, various types of position sensors may be used. For example, in certain embodiment, a non-optical encoder may be used where a voltage level or polarity may be adjusted as a function of encoder signal feedback to achieve a desired angle, speed, or force.

Certain embodiments may relate to a machine-readable medium (e.g., computer readable media) or computer program products that include program instructions and/or data (including data structures) for performing various computer-implemented operations. A machine-readable medium may be used to store software and data which causes the apparatus to perform methods of the present disclosure. The above-mentioned machine-readable medium may include any suitable medium capable of storing and transmitting information in a form accessible by processing device, for example, a computer. Some examples of the machine-readable medium include, but not limited to, magnetic disc storage such as hard disks, floppy disks, magnetic tapes. It may also include a flash memory device, optical storage, random access memory, etc. The data and program instructions may also be embodied on a carrier wave or other transport medium. Examples of program instructions include both machine code, such as produced by a compiler, and files containing higher level code that may be executed using an interpreter.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform or control performing of any of the steps, including but not limited to: displaying, communicating with the user, analyzing, modifying parameters (including timing, frequency, intensity, etc.), determining, alerting, or the like. In some exemplary embodiments hardware may be used in combination with software instructions to implement the present disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “mounted”, “connected”, “attached” or “coupled” to another feature or element, it can be directly mounted, connected, attached or coupled to the other feature or element or intervening features or elements may be present. In contrast, when a feature or element is referred to as being “directly mounted”, “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present apparatuses and methods.

The terms “comprises” and/or “comprising,” when used in this specification (including the claims), specify the presence of stated features, steps, operations, elements, and/or components, but do not preclude the presence or addition of one or more other features, steps, operations, elements, components, and/or groups thereof. Unless the context requires otherwise, “comprise”, and variations such as “comprises” and “comprising,” means various components can be co-jointly employed in the methods and articles (e.g., compositions and apparatuses including device and methods) For example, the term “comprising” will be understood to imply the inclusion of any stated elements or steps but not the exclusion of any other elements or steps.

Any of the apparatuses and methods described herein may include all or a sub-set of the components and/or steps, and these components or steps may be either non-exclusive (e.g., may include additional components and/or steps) or in some variations may be exclusive, and therefore may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and apparatus embodiments may be included in some embodiments and not in others. Therefore, the foregoing description is provided primarily for exemplary purposes and should not be interpreted to limit the scope of the apparatuses and methods as it is set forth in the claims.

Various embodiments may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1.-49. (canceled)

50. An apparatus for delivering a pulsed electric field, the apparatus comprising:
a handle;
an elongate shaft extending from the handle; and
a tip region extending distally from the elongate shaft, the tip region comprising:
a first electrode,
a second electrode distal to the first electrode, wherein the second electrode has a hybrid configuration comprising a distal cutting region and a proximal non-cutting region,
wherein the distal cutting region has one or more blade edges extending proximally and configured to cut tissue, and wherein the proximal non-cutting region has a smooth face extending proximally from a proximal end of each of the one or more blades and configured to stretch and expand tissue; and
a spacer between the first electrode and the second electrode.

51. The apparatus of claim 50, wherein the first electrode and the second electrode are configured to apply sub-microsecond pulses.

52. The apparatus of claim 50, wherein the second electrode is configured to increase hoop stress.

53. The apparatus of claim 50, wherein a circumference of the distal cutting region is about 5% to about 50% of a full circumference of the second electrode at the proximal non-cutting region.

54. The apparatus of claim 50, wherein the proximal non-cutting region of the second electrode comprises a flat or curved conical surface.

55. The apparatus of claim 50, wherein the second electrode is configured to avoid abrupt changes in an angle between walls of the tip region and tissue when the tip region is inserted into the tissue.

56. The apparatus of claim 50, wherein the one or more blade edges comprises three or more blade edges.

57. The apparatus of claim 50, further comprising a vacuum channel configured to provide a negative pressure at the tip region and/or an infusion channel configured to deliver a solution from the tip region.

58. The apparatus of claim 50, wherein a circumference of the spacer at least along a portion of a length of the spacer is greater than a circumference of either of the first electrode or the second electrode.

59. The apparatus of claim 50, further comprising a length adjuster configured to adjust a distance between the first electrode and the second electrode.

60. The apparatus of claim 50, wherein a distance between the first electrode and the second electrode is adjustable from 1 mm to 7 mm.

61. The apparatus of claim 50, further comprising a handle including an insulating baffle configured to provide a minimum clearance distance between electrical contacts for the first electrode and the second electrode.

62. The apparatus of claim 50, wherein each of the first electrode and the second electrode comprises a curved edge on each of a side of the first electrode and the second electrode facing the spacer.

63. The apparatus of claim 50, wherein the spacer is a conductive spacer.

64. The apparatus of claim 50, wherein a maximum circumference of the spacer is greater than a circumference of the first electrode and of the second electrode, and wherein each end of the spacer adjacent to the first electrode and the second electrode has the same circumference as the circumference of the first electrode and the circumference of the second electrode, further wherein the circumference of the spacer tapers from a middle of the spacer towards each end of the spacer.

65. The apparatus of claim 50, wherein the spacer is configured so that a minimum clearance distance between the first electrode and the second electrode is greater than a minimum distance between the first electrode and the second electrode.

66. The apparatus of claim 50, wherein a change in an angle between each of the one or more blade edges relative to a long axis of the tip region as they transition to the smooth face is less than 20 degrees.

67. A method of treating a target tissue, the method comprising:
inserting percutaneously a tip region of a treatment tool into the target tissue, the tip region comprising a first electrode and a second electrode; and
applying a plurality of electrical pulses having a pulse duration of less than 1000 nanoseconds while reducing peak electric field for a given potential and/or increasing hoop stress on the target tissue to prevent or at least reduce arcing between the first electrode and the second electrode.

68. The method of claim 67, wherein increasing hoop stress comprises cutting tissue with one or more distal cutting edges at a distal end of the tip region and stretching the tissue with a proximal portion of the tip region having a smooth surface with no cutting edges and a larger circumference than the distal end of the tip region.

69. The method of claim 67, wherein reducing the peak electric field comprises using a conductive spacer between the first electrode and the second electrode.

70. The method of claim 67, wherein reducing peak electric field comprises using the first electrode and the second electrode with rounded corners.

71. The method of claim 67, wherein increasing hoop stress comprises using a spacer between the first electrode and the second electrode and wherein a circumference of the spacer is larger than a circumference of each of the first electrode and the second electrode.

72. The method of claim 67, wherein the target tissue is a thyroid nodule.

73. The method of claim 67, wherein the target tissue comprises any of the following: a lesion, a tumor, a nodule, or a growth.

74. The method of claim 73, wherein the lesion, the tumor, the nodule or the growth is on or within a muscular organs, circulatory organs, respiratory organs, abdomen organs, digestive organs, urinary organs, immune system organs, nervous system organs, endocrine organs, reproductive organ, or skeletal organs.
